(19) United States
(12) Patent Application Publication
(10) Pub. No.: US 2010/0004500 A1
(43) Pub. Date: Jan. 7, 2010

(54) SYSTEMS AND METHODS FOR VARYING ELECTROMAGNETIC AND ADJUNCTIVE NEURAL THERAPIES

(76) Inventors: Bradford Evan Gliner, Sammamish, WA (US); Jeffrey Balzer, Allison Park, PA (US); Brad Fowler, Woodinville, WA (US); Leif R. Sloan, Seattle, WA (US)

Correspondence Address: ST. JUDE MEDICAL NEUROMODULATION DIVISION, 6901 PRESTON ROAD, PLANO, TX, 75024, US

(21) Appl. No.: 12/561,950

(22) Filed: Sep. 17, 2009

Related U.S. Application Data
(63) Continuation of application No. 11/344,453, filed on Jan. 30, 2006, now abandoned.

(51) Int. Cl. A61N 2/04

(52) U.S. Cl. 600/12; 600/15

(57) ABSTRACT

Systems and methods for varying electromagnetic and adjunctive neural therapies are disclosed. A method in accordance with one embodiment includes applying electromagnetic signals to a target neural population of a patient over a first period of time in accordance with a first mode (e.g., including signal delivery to the central nervous system or peripheral nervous system, via implanted or non-implanted devices). The method can further include applying electromagnetic stimulation to the patient over a second period of time in accordance with a second mode different than the first mode. Varying the mode between the first period of time and second period of time can increase the efficacy and/or longevity of the stimulation. Systems in accordance with other embodiments can support multiple signal delivery devices.

### CROSS-REFERENCE TO RELATED APPLICATIONS

This application is a continuation of U.S. application Ser. No. 11/344,453, filed Jan. 30, 2006, which is incorporated herein by reference.

### TECHNICAL FIELD

The present disclosure is directed generally toward systems and methods for applying, adjusting, or varying electromagnetic and adjunctive neural therapies.

### BACKGROUND

A wide variety of mental and physical processes are controlled or influenced by neural activity in particular regions of the brain. For example, the neural functions in some areas of the brain (i.e., the sensory or motor cortices) are organized according to physical or cognitive functions. Several areas of the brain appear to have distinct functions in most individuals. In the majority of people, for example, the areas of the occipital lobes relate to vision, the regions of the left inferior frontal lobes relate to language, and particular regions of the cerebral cortex appear to be consistently involved with conscious awareness, memory, and intellect.

Many problems or abnormalities can be caused by damage, disease and/or disorders in the brain. Effectively treating such abnormalities may be very difficult. For example, a stroke is a common condition that damages the brain. Strokes are generally caused by emboli (e.g., obstruction of a vessel), hemorrhages (e.g., rupture of a vessel), or thrombi (e.g., clotting) in the vascular system of a specific region of the brain. Such events generally result in a loss or impairment of a neural function (e.g., neural functions related to facial muscles, limbs, speech, etc.). Stroke patients are typically treated using various forms of physical therapy to rehabilitate the loss of function of a limb or another affected body part. Stroke patients may also be treated using physical therapy plus an adjunctive therapy, such as amphetamine treatment. For most patients, however, such treatments are minimally effective and little can be done to improve the function of an affected body part beyond the recovery that occurs naturally without intervention. As a result, many types of physical and/or cognitive deficits that remain after treating neurological damage or disorders are typically considered permanent conditions that patients must manage for the remainder of their lives.

Neurological problems or abnormalities are often related to electrical and/or chemical activity in the brain. Neural activity is governed by electrical impulses or “action potentials” generated in neurons and propagated along synaptically connected neurons. When a neuron is in a quiescent state, it is polarized negatively and exhibits a resting membrane potential typically between −70 and −60 mV. Through chemical connections known as synapses, any given neuron receives excitatory and inhibitory input signals or stimuli from other neurons. A neuron integrates the excitatory and inhibitory input signals it receives, and generates or fires a series of action potentials when the integration exceeds a threshold potential. A neural firing threshold, for example, may be approximately −55 mV.

It follows that neural activity in the brain can be influenced by electrical energy supplied from an external source such as a waveform generator. Various neural functions can be promoted or disrupted by applying an electrical current to the cortex or other region of the brain. As a result, researchers have attempted to treat physical damage, disease and disorders in the brain using electrical or magnetic stimulation signals to control or affect brain functions.

Transcranial electrical stimulation (TES) is one such approach that involves placing an electrode on the exterior of the scalp and delivering an electrical current to the brain through the scalp and skull. Another treatment approach, transcranial magnetic stimulation (TMS), involves producing a magnetic field adjacent to the exterior of the scalp over an area of the cortex. Yet another treatment approach involves direct electrical stimulation of neural tissue using implanted electrodes.

The neural stimulation signals used by these approaches may comprise a series of electrical or magnetic pulses that can affect neurons within a target neural population. Stimulation signals may be defined or described in accordance with stimulation signal parameters, including pulse amplitude, pulse frequency, duty cycle, stimulation signal duration, and/or other parameters. Electrical or magnetic stimulation signals applied to a population of neurons can depolarize neurons within the population toward their threshold potentials. Depending upon stimulation signal parameters, this depolarization can cause neurons to generate or fire action potentials. Neural stimulation that elicits or induces action potentials in a functionally significant proportion of the neural population to which the stimulation is applied is referred to as supra-threshold stimulation; neural stimulation that fails to elicit action potentials in a functionally significant proportion of the neural population is defined as sub-threshold stimulation. In general, supra-threshold stimulation of a neural population triggers or activates one or more functions associated with the neural population, but sub-threshold stimulation by itself does not trigger or activate such functions. Supra-threshold neural stimulation can induce various types of measurable or monitorable responses in a patient. For example, supra-threshold stimulation applied to a patient's motor cortex can induce muscle fiber contractions in an associated part of the body.

More recently, direct cortical stimulation has been used to produce therapeutic, rehabilitative, and/or restorative neural activity, as disclosed in pending U.S. application Ser. No. 09/802,808 Ser. No. 10/606,202, both assigned to the assignee of the present application, and both incorporated herein by reference. These techniques have been used to produce long lasting benefits to patients suffering from a variety of neural disorders. While these techniques have been efficacious, there is a continued need to improve the applicability of these methods to a wide variety of patients, and to further enhance the longevity of the effects produced by these methods.

### BRIEF DESCRIPTION OF THE DRAWINGS

FIG. 1A is a schematic illustration of neurons.

FIG. 1B is a graph illustrating firing an “action potential” associated with normal neural activity.

FIG. 1C is a flowchart of a method for effectuating a neural function of a patient in accordance with one embodiment of the invention.

FIG. 2 is a top plan image of a portion of a brain illustrating neural activity in first and second regions of the brain associated with the neural function of the patient according to the somatotopic organization of the brain.

FIG. 3 is a top plan image of a portion of the brain illustrating a loss of neural activity associated with the neural function of the patient used in one stage of a method in accordance with an embodiment of the invention.

FIG. 4 is a top plan image of the brain of FIG. 3 showing a change in location of the neural activity associated with the neural function of the patient at another stage of a method in accordance with an embodiment of the invention.

FIGS. 5A and 5B are schematic illustrations of an implanting procedure at a stage of a method in accordance with an embodiment of the invention.

FIG. 5C is a graph illustrating firing an “action potential” associated with stimulated neural activity in accordance with one embodiment of the invention.

FIG. 6 is a flow diagram illustrating a method for varying modes of a patient's treatment program in accordance with an embodiment of the invention.

FIG. 7 is a flow diagram illustrating a method for varying adjunctive therapy parameters in accordance with another embodiment of the invention.

FIG. 8A is an isometric illustration of an implantable signal delivery apparatus configured in accordance with an embodiment of the invention.

FIG. 8B is a cross-sectional view of a signal delivery apparatus implanted in accordance with an embodiment of the invention.

FIG. 8C illustrates a system configured to control electrical signals in accordance with an embodiment of the invention.

FIG. 8D illustrates an external controller configured to transmit pulses to electrodes in accordance with an embodiment of the invention.

FIG. 9A is a schematic illustration of a system that includes a controller configured to direct neural therapy signals to different signal delivery devices in accordance with still another embodiment of the invention.

FIG. 9B-9E illustrate systems that include combinations of signal delivery devices configured in accordance with further embodiments of the invention.

FIGS. 10A-10D illustrate power sources and signal delivery devices configured in accordance with embodiments of the invention.

FIG. 11 illustrates an electrode having a “peg” type configuration in accordance with a further embodiment of the invention.

FIGS. 12A-12B illustrate signal delivery devices having multiple electrodes arranged in an array and carried by a single substrate in accordance with further embodiments of the invention.

FIG. 13 illustrates a signal delivery device configured to carry multiple electrodes in accordance with another embodiment of the invention.

FIG. 14 illustrates electrodes having different penetration depths and carried by a single substrate in accordance with still another embodiment of the invention.

FIG. 15 illustrates an electrode configured for deep brain stimulation in accordance with another embodiment of the invention.

FIG. 16 illustrates a method for stimulating neural tissue via transcranial direct current stimulation in accordance with an embodiment of the invention.

FIG. 17 illustrates a method for stimulating neural tissue in transcranial magnetic stimulation in accordance with another embodiment of the invention.

FIG. 18 illustrates electrodes configured to stimulate the vagal nerve in accordance with another embodiment of the invention.

### DETAILED DESCRIPTION

The following disclosure describes several methods and systems for providing electromagnetic signals to treat or otherwise effectuate a change in a neural function of a patient. Several embodiments of methods and systems described herein are directed toward enhancing or otherwise inducing neuroplasticity to effectuate a particular neural function. Neuroplasticity refers to the ability of the brain to change or adapt over time. It was once thought that adult brains became relatively “hard wired” such that functionally significant neural networks could not change significantly over time or in response to injury. It has become increasingly more apparent that these neural networks can change and adapt over time so that meaningful function can be restored or developed in response to neurologic dysfunction such as brain injury. An aspect of several embodiments of methods and systems in accordance with the invention is to facilitate or provide the appropriate triggers for adaptive, restorative, and/or compensatory neuroplasticity. These appropriate triggers appear to cause or enable improved functional signaling capabilities within significant populations of neurons in a network.

Neural signals (e.g., stimulation signals) applied or delivered in various manners described herein may affect the excitability of a portion of a neural network involved in or associated with a functionally significant activity or task such that a selected population of neurons can become more strongly associated with that network. Because such a network will subserve a functionally meaningful activity or process (e.g., motor learning, cognition, processing emotional information/maintaining emotional state, or memory formation/consolidation), neurofunctional changes are more likely to be lasting because they are reinforced by natural use mechanisms. The nature of the stimulation in accordance with various embodiments of the invention may increase a likelihood that a stimulated population of neurons communicates with or links to other neurons in a functional network. In some embodiments, this may occur because action potentials are not actually caused or generally caused by the stimulation itself, but rather the action potentials are caused by interactions with other neurons in the network. Several aspects of the electromagnetic stimulation in accordance with selected embodiments of the invention increase the probability of restoring or developing neural functionality when the network is activated by a combination of electromagnetic stimulation and one or more favorable activities or processes. Such activities may comprise one or more types of behavioral therapy, for example, rehabilitation, limb use, cognitive behavioral therapy, an activity of daily living, or observation of other individuals performing relevant activities.

Various methods in accordance with embodiments of the invention can be used to treat particular symptoms in patients experiencing neurologic dysfunction arising from neurological damage, neurologic disease, neurodegenerative conditions, neuropsychiatric disorders, neuropsychological (e.g., cognitive or learning) disorders, and/or other conditions. Such neurologic dysfunction and/or conditions may correspond to Parkinson's Disease, essential tremor, Huntington's disease, stroke, traumatic brain injury, Cerebral Palsy, Multiple Sclerosis, a central and/or peripheral pain syndrome or condition, a memory disorder, dementia, Alzheimer's disease, an affective disorder, depression, bipolar disorder, anxiety, obsessive/compulsive disorder, Post Traumatic Stress Disorder (PTSD), an eating disorder, schizophrenia, Tourette's Syndrome, Attention Deficit Disorder, dyslexia, a phobia, an addiction (e.g., alcoholism or substance abuse), autism, epilepsy, a sleep disorder (e.g., sleep apnea), an auditory disorder (e.g., tinnitus or auditory hallucinations), a language disorder, a speech disorder (e.g., stuttering), migraine headaches, and/or one or more other disorders, states, or conditions. In other embodiments identical or at least generally similar methods and systems can be used to enhance the neural functioning of patients who otherwise function at normal or even above normal levels.

In general, a stimulation site may be defined as an anatomical region, location, or site at which electromagnetic signals (e.g., stimulation signals) may be applied or delivered to the patient. Such signals may be intended to directly and/or indirectly affect one or more target neural populations, for example, by passing or traveling to, into, through, and/or near a target neural population. In various embodiments, one or more stimulation sites and/or target neural populations may reside upon or within one or more cortical regions, for example, a portion of the premotor cortex, the motor cortex, the supplementary motor cortex, the somatosensory cortex, the prefrontal cortex, and/or another cortical region. Additionally or alternatively, one or more stimulation sites and/or target neural populations may reside elsewhere, for example, in a subcortical or deep brain region, within or upon the cerebellum, and/or upon or proximate to portions of the spinal cord and/or one or more cranial or other peripheral nerves.

A target neural population and/or a stimulation site may be identified and/or located in a variety of manners, for example, through one or more procedures involving the identification of anatomical features or landmarks; electrophysiological signal measurement (e.g., electroencephalography (EEG), electromyography (EMG), silent period, coherence, and/or other measurements); neural imaging (e.g., Magnetic Resonance Imaging (MRI), functional MRI (fMRI), Diffusion Tensor Imaging (DTI), Perfusion Weighted Imaging (PWI), Positron Emission Tomography (PET), single photon emission computed tomography (SPECT), optical imaging (e.g., near infrared-spectroscopy (NIRS) or optical tomography (OT)), Magnetoencephalography (MEG), and/or another technique); neurofunctional mapping (e.g., using TMS and/or intraoperative stimulation); vascular imaging (e.g., Magnetic Resonance Angiography (MRA)); chemical species analysis (e.g., Magnetic Resonance Spectroscopy (MRS)); and/or another type of functional and/or structural anatomic assessment technique (e.g., Transcranial Doppler ultrasonography (TCD)).

Certain methods in accordance with embodiments of the invention electrically and/or magnetically stimulate the brain at a stimulation site where neuroplasticity is occurring or has occurred, and/or where neuroplasticity is expected to occur. In particular embodiments, the manner in which the electromagnetic signals are applied to the brain and/or other neural tissue can be varied over the course of two or more time periods. For example, a type of signal source and/or a waveform, amplitude, pulse pattern, and/or location at which stimulation is applied can be varied from one time period to the next. In still further embodiments, the manner in which one or more adjunctive therapies are applied during a therapy program can be varied from one time period to another. For example, a type of behavioral therapy and/or a manner in which a patient undergoes such therapy can be varied. The adjunctive therapy can occur simultaneously with the electromagnetic stimulation, or at other times, depending upon the patient's condition.

Other aspects of the invention are directed to systems that support different modes via which electromagnetic signals are applied to the patient. For example, a system in accordance with one aspect of the invention includes a controller that is coupleable to at least two different kinds of signal delivery devices. The controller can provide electromagnetic stimulation in accordance with different modes, depending upon which device it is coupled to. The signal delivery devices can be selected to include (for example) implanted cortical electrodes, subcortical or deep brain electrodes, cerebellar electrodes, spinal column electrodes, vagal nerve (or other cranial or peripheral nerve) electrodes, transcranial electrodes and/or transcranial magnetic stimulators.

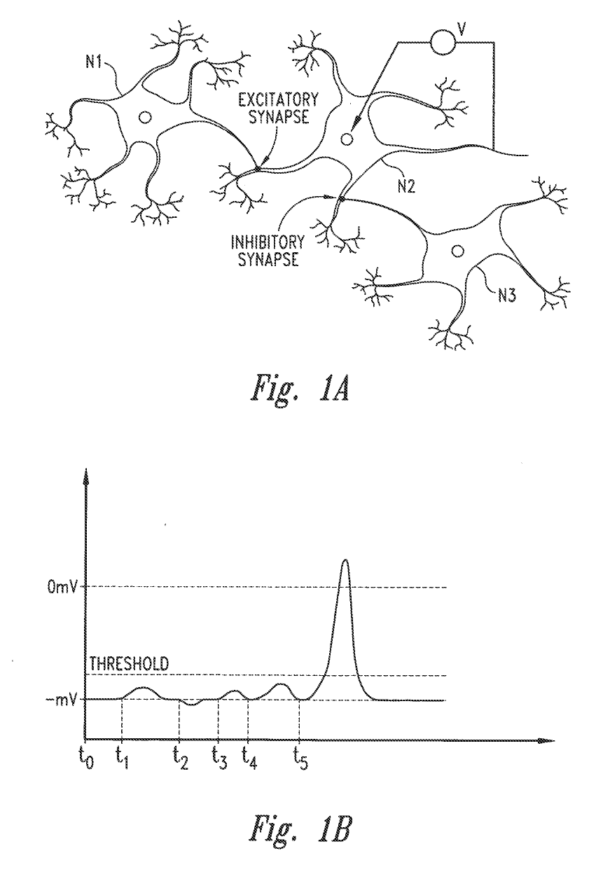

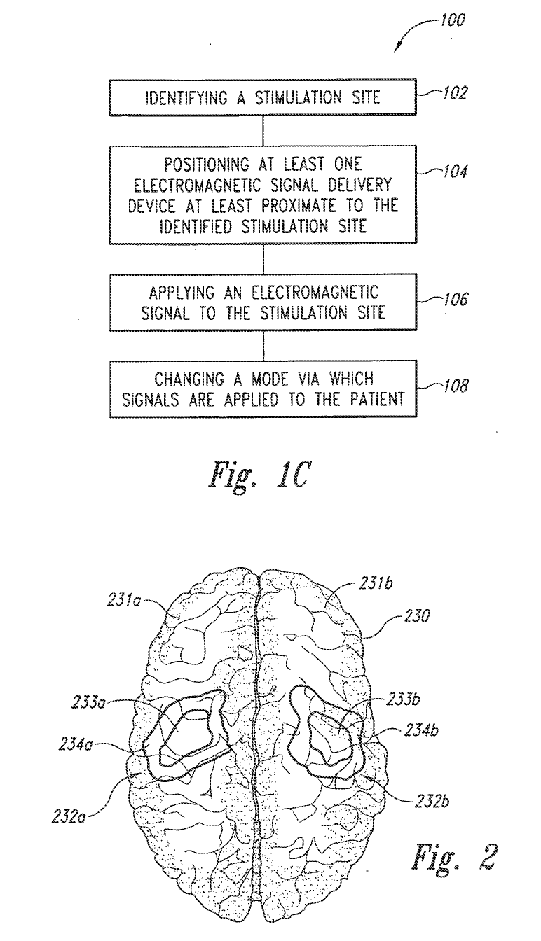

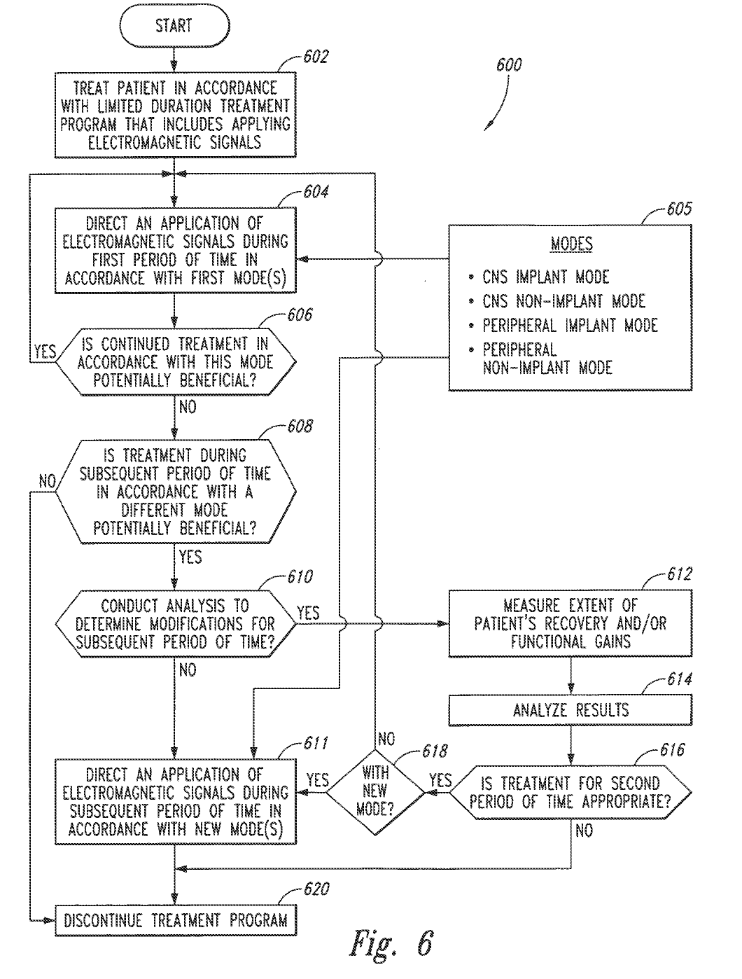

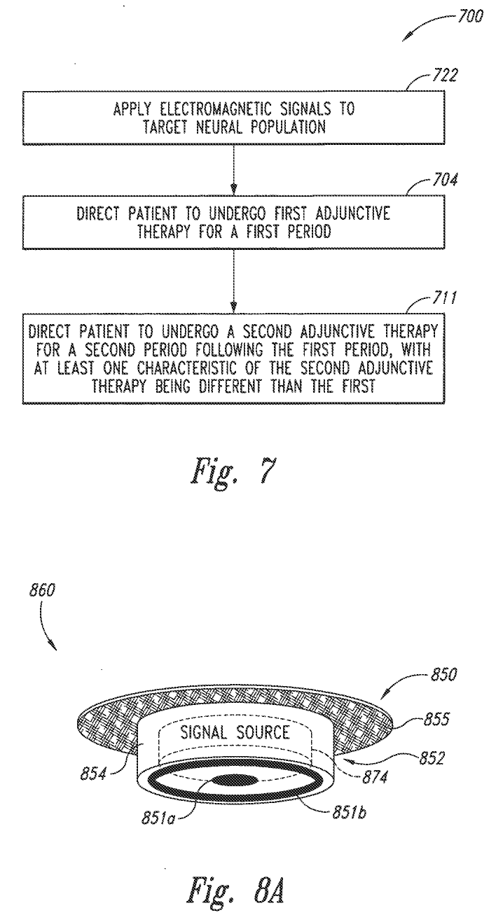

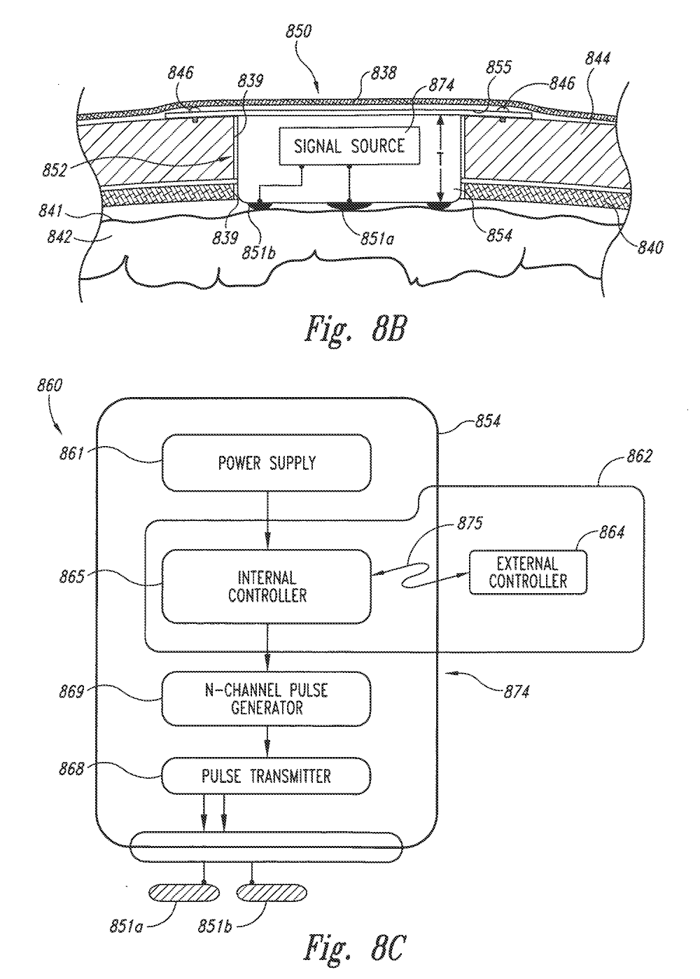

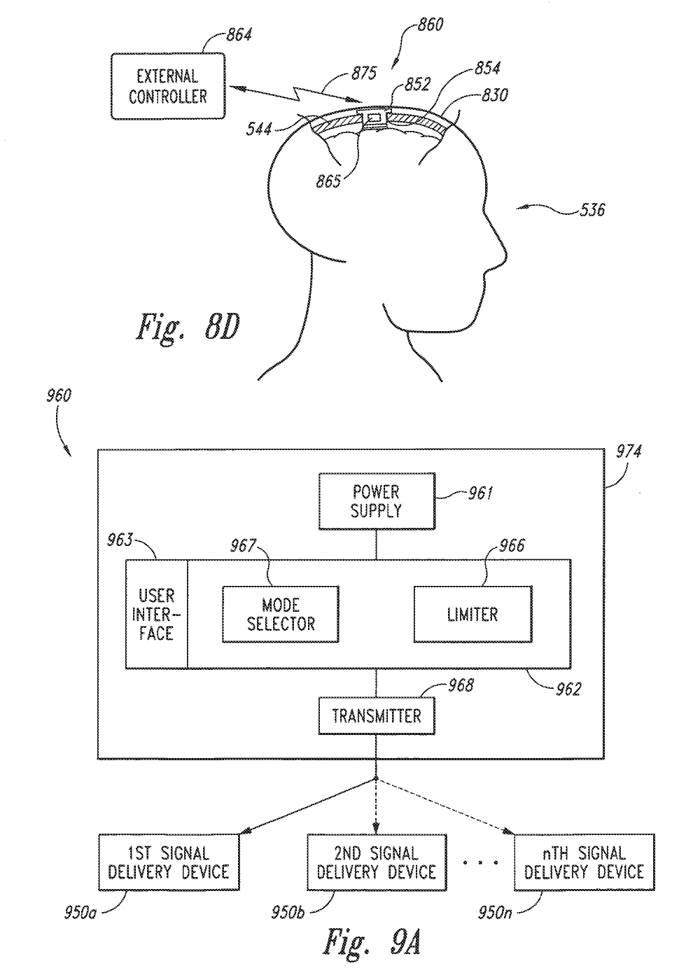

The specific details of certain embodiments of the invention are set forth in the following description and in FIGS. 1A-18 to provide a thorough understanding of these embodiments to a person of ordinary skill in the art. More specifically, several methods and systems in accordance with embodiments of the invention are initially described with reference to FIGS. 1A-5C. More specific examples of such methods are described with reference to FIGS. 6-7. Systems for providing electromagnetic stimulation in accordance with different modes are further described with reference to FIGS. 8A-18. A person skilled in the relevant art will understand that the present invention may have additional embodiments, and that the invention can be practiced without several of the details described below.

### A. Overall Systems And Methods

FIG. 1A is a schematic representation of several neurons N1-N3 and FIG. 1B is a graph illustrating an “action potential” related to neural activity in a normal neuron. Neural activity is governed by electrical impulses generated in neurons. For example, neuron N1 can send excitatory inputs to neuron N2 (e.g., at times to, t3 and t4 in FIG. 1B), and neuron N3 can send inhibitory inputs to neuron N2 (e.g., at time t2 in FIG. 1B). The neurons receive/send excitatory and inhibitory inputs from/to a population of other neurons. The excitatory and inhibitory inputs can produce “action potentials” in the neurons, which are electrical pulses that travel through neurons by changing the flux of sodium (Na) and potassium (K) ions across the cell membrane. An action potential occurs when the resting membrane potential of the neuron surpasses a threshold level. When this threshold level is reached, an “all-or-nothing” action potential is generated. For example, as shown in FIG. 1B, the excitatory input at time t5 causes neuron N2 to “fire” an action potential because the input exceeds the threshold level for generating the action potential. The action potentials propagate down the length of the axon (the long portion of the neuron that makes up nerves or neuronal tracts) to cause the release of neurotransmitters from that neuron that will further influence adjacent neurons.

FIG. 1C is a flowchart illustrating a method 100 for facilitating and/or effectuating a neural function in a patient in accordance with an embodiment of the invention. The neural function, for example, can control a specific mental process or physiological function, such as a particular motor function (e.g., movement of a limb) or sensory function that is normally associated with neural activity at a “normal” location in the brain according to the functional organization of the brain. In several embodiments of the method 100, at least some neural activity related to the neural function can be occurring at one or more sites in the brain. A site associated with the neural activity may involve one or more portions of a normal location where neural activity typically occurs or is expected to occur to carry out the neural function according to the functional organization of the brain, and/or a site associated with the neural activity may be at a different location where the brain has recruited material to perform the neural activity. In either situation, one aspect of several embodiments of the method 100 is to determine or otherwise identify the location(s) in the brain where this neural activity is present and/or expected.

The method 100 may include a diagnostic procedure 102 involving identifying at least one stimulation site corresponding to an anatomical location at which stimulation signals may be applied or delivered to one or more target neural populations. In various embodiments, such neural populations may reside within the central nervous system, and in particular embodiments, one or more target neural populations may reside within the brain. In some embodiments, particular target neural populations may include one or more portions of the peripheral nervous system.

In one approach, a set of stimulation sites may be particular locations of the brain and/or the spinal cord where an intended neural activity related to a given type of neural function is present or is expected to be present. For example, the stimulation site may be particular neural regions and/or cortical structures that are expected to direct, effectuate, and/or facilitate specific neural functions in most individuals. In another approach, the stimulation site may be a location of the brain that supports or is expected to support the intended neural function.

The diagnostic procedure 102 may include identifying one or more anatomical landmarks on the patient that correspond to such neural populations, regions, and/or structures. The anatomical landmarks serve as reference points for identifying or approximately identifying a neural location (e.g., a brain or spinal cord location) where an intended neural activity may occur. Thus, one aspect of the diagnostic procedure 102 may include referencing a stimulation site relative to anatomical landmarks. More specifically, identifying an anatomical landmark may include visually determining the location of one or more reference structures (e.g., visible cranial landmarks), and locating underlying brain regions or structures (e.g., the motor strip and/or the Sylvian fissure) relative to the external location of the reference structures. Such reference structures may include, for example, the bregma, the midsagittal suture, and/or other well-known cranial or other landmarks referenced in a manner understood by those skilled in the art. The methods for locating an underlying brain structure typically involve measuring distances and angles relative to the cranial topography, as is known in the art of neurosurgery.

The diagnostic procedure 102 may additionally or alternatively include identifying one or more enhanced-precision or patient-specific stimulation sites and/or target neural populations. A patient-specific stimulation site may be identified in various manners, including one or more of MRI, fMRI, DTI, MRS, MRA, PET, SPECT, MEG, NIRS, OT, EEG, intraoperative mapping, and/or another technique capable of localizing, measuring, or monitoring neuroanatomical structures, neurofunctional or neurometabolic activity or activity correlates, and/or chemical species concentrations.

In one embodiment, the diagnostic procedure 102 includes identifying, generating, or characterizing an intended neural activity in the brain at a supplementary, auxiliary, derivative, secondary, or peripheral location that is different, distinct, or remote from a normal location, and determining where the intended neural activity is actually present in the brain. In an alternative embodiment, the diagnostic procedure 102 can be performed by identifying a stimulation site where neural activity has changed in response to a change in the neural function.

The method 100 continues with a positioning procedure 104 involving positioning at least one electromagnetic signal delivery device or signal transfer element relative to an identified stimulation site, and a stimulating procedure 106 involving applying an electromagnetic signal to the signal delivery device. Several embodiments of the positioning procedure 104 include positioning two or more electrodes at a stimulation site (e.g., in a bipolar arrangement), but other embodiments of the implanting procedure involve positioning only one electrode at a stimulation site and another electrode remotely from the stimulation site (e.g., in a unipolar arrangement). In still further embodiments, stimulation can be applied without implanting electrodes (e.g., by delivering stimulation transcranially). Particular embodiments include changing the signal delivery mode (e.g., the type of signal delivery device and/or the location to which signals are directed) during the course of a treatment regimen (process portion 108).

FIGS. 2-4 illustrate specific embodiments of the diagnostic procedure 102. A diagnostic procedure 102 can be used to determine one or more regions of the central nervous system where stimulation will likely facilitate or effectuate a desired result, such as rehabilitating a malfunction in or degradation or loss of a neural function caused by a stroke, trauma, disease or other circumstance. FIG. 2, more specifically, is an image of a normal, healthy brain 230 having a first region 232a in a first hemisphere 231a where an intended or normal neural activity occurs to effectuate a specific neural function in accordance with the functional organization of the brain. The first region 232a can have a high-intensity area 233a and/or a low-intensity area 234a at which different levels of neural activity occur. It is not necessary to obtain an image of the neural activity in the first region 232a shown in FIG. 2 to carry out the diagnostic procedure 102, but rather it is provided to show an example of neural activity that typically occurs at a “normal location” according to the functional organization of the brain 230 for a large percentage of people with normal brain function.

The brain 230 of FIG. 2 also indicates neural activity in a second region 232b, which may reside within in a second hemisphere 231b of the brain. The actual location of the first and/or second regions 232a, 232b may vary somewhat between individual patients, but those skilled in the art will recognize that such locations will bear a fairly predictable spatial relationship with respect to anatomical features of the patient's skull for a majority of individuals. In general, each hemisphere 231a, 231b of the brain 230 is responsible for exerting primary or majority control over motor and/or sensory functions on the opposing or “contralateral” side of the patient's body. For example, the neural activity in the first region 232a shown in FIG. 2 may be generally associated with the movement of fingers on a patient's right hand, whereas the second region 232b in the right hemisphere 231b may be generally associated with movement of fingers on the patient's left hand. This second region 232b, like the first region 232a, may have a high-intensity area 233b and a low-intensity area 234b in which different levels of neural activity related to movement of the patient's left-hand fingers occur. The first region 232a may be associated with a body part or parts (in this example, the fingers of the right hand) and the second region 232b may be associated with a contralateral homotypic body part (in this case, the fingers of the left hand), i.e., another body part having the same or an analogous structure or function as, but contralateral to, the first body part. This is one example of a body function (movement of the left fingers) that may be a corollary to another body function (movement of the right fingers).

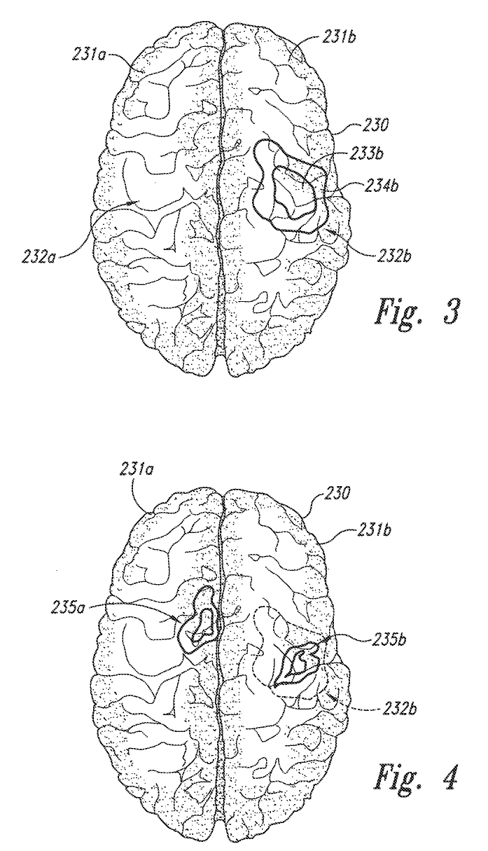

The neural activity in the first region 232a, however, can be impaired. In one embodiment, the diagnostic procedure 102 begins by taking an image of the brain 230 that is capable of detecting neural activity to determine whether the intended neural activity associated with the particular neural function of interest is occurring at the region of the brain 230 where it normally occurs according to the functional organization of the brain, and/or in a manner in which it would normally be expected to occur. FIG. 3 is a representative image of the brain 230 after the first region 232a has been affected (e.g., from a stroke, trauma or other cause). As shown in FIG. 3, the neural activity that controlled the neural function for moving the fingers of the right hand no longer occurs in the first region 232a. The first region 232a is thus “inactive,” which is expected to result in a corresponding loss of the movement and/or sensation in the fingers. In some instances, the damage to the brain 230 may result in only a partial loss of the neural activity in the damaged region. In either case, the image shown in FIG. 3 establishes that the loss of the neural function is related to the diminished neural activity in the first region 232a. The brain 230 may accordingly recruit other neurons to perform neural activity for the affected neural function (e.g., via neuroplasticity), or the neural activity may not be present at any location in the brain. As suggested in FIG. 3, a corollary neural function associated with the contralateral homotypic body part (in this case, movement of the fingers of the left hand), which is associated with the second region 232b, may remain largely unimpaired. It is worth noting that the second region 232b associated with the corollary body function is at a contralateral homotypic location to the first region 232a, i.e., the location of the second region 232b on the second hemisphere 231b is homologous or generally corresponds to the location of the second region 232a on the first hemisphere 231a.

FIG. 4 is an image of the brain 230 illustrating a plurality of potential stimulation sites 235a and 235b for effectuating the neural function that was originally performed in the first region 232a shown in FIG. 2. It is worth noting that the first potential stimulation site 235a is in the same hemisphere 231a as the first region 232a shown in FIG. 2. Because this first stimulation site 235a is on the same side of the body as the first region 232a, it may be referred to as being “ipsilateral” to the first region 232a. As the first region 232a in the left hemisphere 231a of the brain 230 controls movement on the right side of the body, this first potential stimulation site 235a also may be said to be contralateral to the body function impaired by the inactive status of the first region 232a. The second potential stimulation site 235b, in contrast, is in the right hemisphere 231b of the brain 230 and is therefore contralateral to the first region 232a and ipsilateral to the impaired body function associated with the first region 232a.

The two hemispheres 231a and 231b of the brain 230 are connected via the corpus callosum, which facilitates information transfer between the hemispheres. Although each hemisphere 231a, 231b generally exerts majority control over motor and/or sensory functions on the opposite or contralateral side of the patient's body, each hemisphere typically also exerts some level of control and/or influence over motor and/or sensory functions on the same or ipsilateral side of the patient's body. Moreover, through transcallosal connections, neural activity in one hemisphere may influence or modulate neural activity, e.g., neuroplasticity, in the opposite hemisphere. The location in the brain 230 that exerts influence on an ipsilateral body function frequently is proximate to or subsumed within the location of the brain associated with a corollary body function. Hence, as suggested in FIG. 4, the second potential stimulation site 235b, which is ipsilateral to the body function associated with the inactive first region 232a, may lie within the second region 232b of the brain. As discussed above in connection with FIG. 2, this second region 232b may be associated with a corollary to the impaired body function. In the particular example mentioned above wherein the first region 232a (which resides within the left hemisphere 231a) is associated with movement of the fingers of the patient's right hand, the second potential stimulation site 235b may be positioned proximate to or within a region of the brain (i.e., the second region 232b, which resides within the right hemisphere 231b) associated with movement of the contralateral homotypic body part, namely the fingers of the patient's left hand.

The stimulation sites can be characterized as ipsilateral or contralateral, with reference to particular brain regions or body functions, as described above. In some instances, it may be useful to describe the stimulation sites with reference to an affected neural population. In such instances “ipsilesional” is used to refer to a site that is at the same hemisphere as an affected neural population, and “contralesional” is used to refer to a site that is at the opposite hemisphere as the affected neural population, whether the affected neural population is affected by a lesion or another condition. Either set of terms may be used herein to characterize the site, depending upon the particular context.

The diagnostic procedure 102 may utilize evidence of a set of neural structures, a level of neural activity, neuroplasticity, and/or chemical species information within the brain to identify the location of a stimulation site that is expected to be more responsive to the results of an electrical, magnetic, sonic, genetic, biologic, pharmaceutical, mechanical, thermal, or other procedure to facilitate or effectuate a desired neural function. One embodiment of the diagnostic procedure 102 involves measuring, estimating, or characterizing types or levels of neural activity or chemical species in particular brain regions relative to other (e.g., corollary) brain regions, a set of reference brain regions (e.g., corresponding to a population of healthy individuals), and/or different time periods.

Another embodiment of the diagnostic procedure 102 involves generating an intended neural activity remotely from the first region 232a of the brain, and then detecting or sensing the location(s) in the brain where the intended neural activity has been generated. The intended neural activity can be generated by causing a signal to be generated within and/or sent to the brain. For example, in the case of a patient having an impaired limb, the affected limb is moved and/or stimulated while the brain is scanned using a known imaging technique that can detect neural activity (e.g., fMRI, PET, etc.). In one specific embodiment, the affected limb can be moved by a practitioner or the patient, stimulated by sensory tests (e.g., pricking), or subjected to peripheral electrical stimulation. In another embodiment, the patient can attempt to move the affected limb, or imagine or visualize moving the affected limb in one or more manners. The attempted or imagined movement/actual movement/stimulation of the affected limb produces a neural signal corresponding to the limb (e.g., a peripheral neural signal) that is expected to generate a response neural activity in the brain. The location(s) in the brain where this response neural activity is present can be identified using the imaging technique. FIG. 4, for example, can be created by moving, attempting to move, or visualizing the movement of the affected fingers and then noting where neural activity occurs in response. By generating an intended neural activity in such a manner, this embodiment may accurately identify where the brain has recruited matter (i.e., sites 235a and 235b) to perform the intended neural activity associated with the neural function.

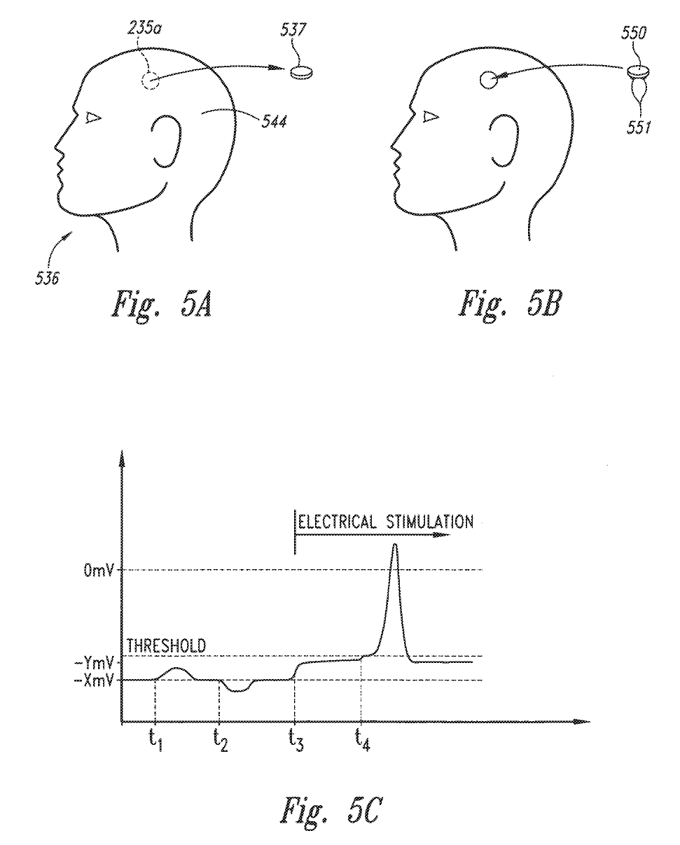

FIGS. 5A and 5B are schematic illustrations of a particular embodiment of the positioning procedure 104 described above with reference to FIG. 1C. In this embodiment, positioning includes implanting one or more electrodes relative to a portion of the brain of a patient 536. Such electrodes may be implanted epidurally or subdurally. Referring to FIG. 5A, the stimulation site 235a is identified in accordance with an embodiment of the diagnostic procedure 102. In one embodiment, a skull section 537 is removed from the patient 536 adjacent to the stimulation site 235a. The skull section 537 can be removed by boring a hole in the skull 544 in a manner known in the art, or a much smaller hole can be formed in the skull 544 using drilling techniques that are also known in the art. Referring to FIG. 5B, an implantable signal delivery device 550 coupled to or carrying at least a first and possibly a second or additional electrodes 551 can be implanted in the patient 536. Suitable techniques associated with the implantation procedure are known to practitioners skilled in the art. After the signal delivery device 550 has been implanted in the patient 536, a pulse system generates electrical pulses that are transmitted to the stimulation site 535a by the first and/or second electrodes 551. Signal delivery devices suitable for carrying out the foregoing methods in accordance with embodiments of the invention are described in more detail later with reference to FIGS. 8A-18. The positioning procedure 104 may also include implanting one or more monitoring devices such as sensing electrodes in the patient 536.

Depending upon embodiment details, subthreshold and/or suprathreshold stimulation signals may be applied to particular stimulation sites. FIG. 5C is a graph illustrating the application of a subthreshold potential to the neurons N1-N3 of FIG. 1A. At times to and t2, the excitory/inhibitory inputs from other neurons do not “bridge-the-gap” from the resting potential at −X mV to the threshold potential. At time t3, the electromagnetic stimulation is applied to the brain to raise the resting potential of neurons in the stimulated population such that the resting potential is at −Y mV. As such, at time t4 when the neurons receive another excitatory input (which may arise from or correspond to a patient activity (e.g., an actual, attempted, or imagined movement) and/or an electromagnetic stimulation signal applied to the central or peripheral nervous systems), even a small input exceeds the gap between the raised resting potential −Y mV and the threshold potential to induce action potentials in these neurons. For example, if the resting potential is approximately −70 mV and the threshold potential is approximately −50 mV, then the electrical stimulation can be applied to raise the resting potential of a sufficient number of neurons to approximately −52 to −60 mV.

Several embodiments of methods for affecting or enhancing neural activity in accordance with the invention are expected to provide lasting results that promote a desired neural function. At least some of these embodiments may also provide lasting results because electromagnetic stimulation therapies described herein may be applied or delivered to a patient in association with or simultaneously with one or more synergistic or adjunctive therapies. Such synergistic or adjunctive therapies may include or involve the patient's performance or attempted performance of one or more behavioral therapies, activities, and/or tasks. Aspects of the electromagnetic therapy and/or the adjunctive therapy can be varied during the course of treatment to extend and/or otherwise enhance the effects of these treatments, as described below.

### B. Methods For Altering Treatment During A Treatment Program

FIG. 6 is a flow diagram illustrating an overall process 600 for addressing neural dysfunction in a patient, and/or otherwise enhancing the neural functioning of the patient. Process portion 602 is directed to treating the patient in accordance with a limited duration treatment program that includes applying electromagnetic signals. In process portion 604, the program includes treating the patient by directing an application of electromagnetic signals to the patient during a first period of time in accordance with a first mode. The first mode can include parameters associated with the manner in which electrical or magnetic (collectively, electromagnetic) signals are applied to the patient. Four representative modes are shown in block 605 as (a) a central nervous system (CNS) implant mode, (b) a CNS non-implant mode, (c) a peripheral implant mode, and (d) a peripheral non-implant mode. CNS modes include modes in which electromagnetic signals are provided to the patient's central nervous system (e.g., the brain, including the cerebrum, cerebral cortex, cerebellum, cerebellar cortex, deep brain structures, brain stem and spinal column). Peripheral modes include modes in which electromagnetic signals are provided to the patient's peripheral nervous system (e.g., cranial nerves (including the vagal nerve), sensory nerves, and other non-CNS nerves). Implant modes include modes in which the electromagnetic signals are delivered from a device implanted in the patient (e.g., an implanted electrode or microstimulator, such as a bionic neuron or BION™, manufactured by Advanced Bionics Corporation of Sylmar, Calif.). Non-implant modes include modes in which the electromagnetic signals are delivered from a signal delivery device that is not implanted. Each of the modes includes directing an application of electromagnetic signals, which can be performed automatically by an appropriately programmed computer readable medium, and/or with patient and/or practitioner involvement in a manual or semi-autonomous arrangement. Signals can be provided to the patient in accordance with multiple modes (e.g., simultaneously) during the first period, and/or during subsequent periods. Further details of devices that provide electromagnetic signals in accordance with these modes are described later with reference to FIGS. 8A-18.

Signals applied in accordance with any of the foregoing modes can optionally be associated with one or more adjunctive therapies in addition to the electromagnetic therapy. As used herein, an adjunctive therapy refers to a therapy that is different than the electromagnetic signals, but is provided in association or conjunction with the electromagnetic signals. For example, an adjunctive therapy can include a behavioral therapy or a drug therapy. The adjunctive therapy may in some cases be provided simultaneously with the electromagnetic signals, and in other cases, may be provided before or after the electromagnetic signals. Further details of specific types of adjunctive therapies are described later with respect to FIG. 7.

In process portion 606, a determination is made as to whether continued treatment in accordance with the current mode (e.g., the first mode or first mode set) is potentially beneficial. If so, the process returns to process portion 604 for additional treatment in accordance with that mode. If not, then in process portion 608, an evaluation is made as to whether treatment during a subsequent (e.g., a second) period of time with a different mode (e.g., a second mode that is different from a first mode, or a second mode set that involves additional or fewer modes than a first mode set), would be potentially beneficial. If it is determined that such a treatment would not be potentially beneficial, the treatment program is discontinued (process portion 620).

If instead it is determined at process portion 608 that treatment during a subsequent period of time with a different mode may be beneficial to the patient, the process 600 can further include determining whether or not to conduct an analysis to determine the modifications to be made for treatment during the subsequent period of time (process portion 610). For example, in some cases, it may be clear, based on past experience and the patient's recovery performance, in what manner the treatment program should be varied during the subsequent time period. In these cases, the process can move directly to process portion 611, which includes directing an application of electromagnetic signals during the subsequent period of time in accordance with a different mode. If it is not immediately clear which mode (or modes) should be adopted during the subsequent time period, the process 600 can move to process portion 612, which includes measuring the extent of the patient's recovery and/or functional gains. This measurement can be made by having the patient perform tests or undergo other diagnostic procedures, in most cases, similar or identical to diagnostic procedures the patient performed before initiating the program in process portion 602. In process portion 614, the results are analyzed. For example, by comparing the results after the patient has completed treatment for the first period of time with results obtained either before treatment or during treatment during the first period of time, a practitioner can identify the progress the patient has made. The practitioner can then review the available alternate modes and select one or more modes expected to provide an enhanced effect when applied during the subsequent period of time.

After completing the analysis in process portion 614, the practitioner can again assess whether treatment for the subsequent period of time is still appropriate (process portion 616). If not, (for example, if the analysis completed in process portion 614 indicates that such treatment would not be beneficial), the program is discontinued (process portion 620). If subsequent treatment is appropriate, the practitioner can determine whether the treatment program should be continued with a new mode or the current mode (process portion 618). For example, if the analysis completed in process portion 614 indicates that in fact continued treatment with the current mode remains appropriate, the process can return to process portion 604. If the analysis confirms that treatment with a new mode is appropriate, the practitioner can treat the patient during the subsequent period of time in accordance with the new mode (process portion 611). In process portion 611, the new mode may be selected from block 605 to be different than a previously used mode.

### 1. Signal Application Parameters

Signal application parameters refer generally to parameters, other than the mode, via which the practitioner can adjust the effect of the signals on the patient. For example, the practitioner can select the signal application parameters to have a facilitatory or an inhibitory effect on a target neural population. The signal parameters selected by the practitioner can include the current level, voltage level, polarity, waveform type, and/or duration or duty cycle of the signals applied to the patient. The current or voltage level can be selected to be a percentage of the patient's threshold response or level for a given target neural population. As described above, a threshold level can correspond to a signal level or magnitude necessary to trigger a motion response, a sensation, or another observable, measurable, or monitorable effect. When the signals are provided in a time-varying manner, the parameters can further include the width of pulses transmitted to the patient, an overall or representative frequency with which signals are transmitted to the patient, and/or a modulation function that identifies or specifies the manner in which the pulses are varied during treatment. Stimulation signals may be periodic or aperiodic (e.g., random, pseudo-random, or chaotic).

The electromagnetic signals described above can be provided over the course of hours, weeks and/or months in accordance with any of several schedules. For example, the electromagnetic signals can be applied during the first period for three hours per day, 3-5 days per week, for 2-8 or 3-6 weeks, via implanted cortical and/or other electrodes. The electromagnetic stimulation portion of the treatment may then be suspended for an intermediate period of time (e.g., several hours, days, weeks, or months) during which the patient may rest or consolidate neurofunctional gains, and/or still undergo adjunctive therapies. The patient may then undergo another stimulation therapy in accordance with another mode (e.g., via transcranial direct current stimulation (tDCS)) for a period of hours, days or weeks (e.g., one hour, twice a week for four weeks) during the second period of time.

Depending upon embodiment details or patient condition, stimulation therapy in accordance with a particular mode or set of modes may be provided over a limited duration time period (e.g., the first period), and stimulation therapy in accordance with a different mode or mode set may be provided over another limited duration time period or an ongoing or essentially permanent time period (e.g., the second period). Stimulation therapy provided in separate time periods may be directed toward identical, similar, or different types of neurologic dysfunction or patient symptoms. As an example, stimulation therapy during a limited duration first time period may be directed toward functional recovery following neurologic damage, and stimulation therapy during a long-term or ongoing second time period may be directed toward alleviating a central pain syndrome. As another example, stimulation during a limited duration first time period may be directed toward treating post-stroke depression (e.g., using TMS and/or tDCS) and/or restoring motor function (e.g., using a set of implanted cortical electrodes), while stimulation during a limited duration second time period may be directed toward restoring motor, language, and/or cognitive functions (e.g., using the same and/or a different set of implanted cortical electrodes).

In any of the foregoing embodiments, the electromagnetic signals may be preceded by or followed by conditioning stimuli. The conditioning stimuli can be provided immediately or nearly immediately before or after the primary therapeutic signals, and can be provided via a different mode. For example, if the primary therapeutic signals are provided by one or more implanted electrodes, the conditioning stimuli can be provided by tDCS or TMS. In particular embodiments, the conditioning stimuli can be provided within minutes or hours of the primary therapeutic signals, during either the first or second period of time. The conditioning stimuli may be provided in the same brain hemisphere as and/or the opposite brain hemisphere of the primary therapeutic stimulation. The conditioning stimuli are expected to enhance and/or preserve the effects of the primary therapeutic stimulation.

The selectable signal parameters can also include the location(s) at which signals are applied. For example, the signals may be applied to different sites of the patient's nervous system during different phases of a treatment regimen. The sites can be selected from at least the following locations: a location above the cerebral cortex, a location at the cerebral cortex, a location below the cerebral cortex, a cerebellar location, a spinal column location, a location proximate to a cranial (e.g., vagal) or other peripheral nerve, and a location proximate to a muscle. The location may also be varied within one of the above location parameters. For example, during one portion of a treatment regimen, the signals may be provided to one position above or at the cerebral cortex (e.g., proximate to the prefrontal cortex or motor cortex within a given brain hemisphere) and during another portion, the signals may be provided to another position, also above or at the cerebral cortex (e.g., proximate to the premotor cortex within the same or the opposite hemisphere).

In some situations, the selection of the target signal site (in addition to the mode via which the signals are delivered) may be influenced by evidence of changes the patient's brain may have undergone during a prior time period. For example, if it is determined that during the first period of time, the patient's brain has begun recruiting neurons at a site different than the site stimulated during the first period of time, then during the subsequent period of time, the location at which stimulation is provided can be adjusted to correlate more closely with the location at which the brain is recruiting neurons. In another example, it may become apparent after stimulating an ipsilesional stimulation site (e.g., a site in the same hemisphere as damaged or dysfunctional brain tissue) for the first period of time that stimulating a contralesional site may be beneficial. In particular, the ipsilesional stimulation may not have the desired effect or level of desired effect. In such a situation, stimulation during the subsequent period of time can be applied to a contralesional portion of the brain (e.g., the corresponding portion of the brain located in the opposite hemisphere), either alone or in combination with applying stimulation to the ipsilesional brain region.

A change in location may include combinations of any of the parameters described above. For example, during the first time period, the patient may be stimulated in the left hemisphere above the cortex, and during the second time period, the patient may be stimulated in the right hemisphere below the cortex. In some cases, the electrodes implanted in the patient's brain and/or other neuroanatomical location prior to the first period of time may be in a position to provide stimulation during the second period of time as well. In other embodiments, additional electrodes may be implanted prior to the second period of time.

In still further embodiments, the stimulation provided during the second period of time may not require implanting new electrodes, even if the electrodes implanted for stimulation during the first period of time are not positioned properly for stimulation during the second period of time. For example, stimulation provided during the second period of time may include transcranial direct current stimulation or tDCS, (discussed further below with reference to FIG. 17) and/or transcranial magnetic stimulation or TMS (discussed further below with reference to FIG. 18). In some cases, these methods may be conducted without regard to the location of particular implanted electrodes. In other cases, it may be advantageous to provide tDCS and/or TMS in locations where electrodes have been implanted, for example, if the presence of the electrodes enhances stimulation to adjacent neural tissue even when electrical current is not provided directly (e.g., via wires) to the electrodes. In still another embodiment, the order in which the signals are applied can be reversed. For example, the signals can be provided transcranially without implanting electrodes during the first period of time and then electrodes can be implanted prior to applying signals during the second period of time. In any of these embodiments, the signal delivery device used to provide the electromagnetic signals may be changed from one time period to the other as part of changing from one mode to another. (e.g., by changing from implanted electrodes to a transcranial magnetic device). In further embodiments, the signal delivery device selected for a particular time period can include other devices, such as a deep brain electrode. Representative devices that deliver stimulation signals in accordance with those modes are described later with reference to FIGS. 8A-18.

### 2. Adjunctive Therapies

FIG. 7 illustrates portions of a process 700 conducted in accordance with another embodiment of the invention. The process can include applying electromagnetic signals to a target neural population of the patient (process portion 722). The process can also include directing the patient to undergo a first adjunctive therapy for a first time period (process portion 704). The first adjunctive therapy can be, but need not be, simultaneous with the application of electromagnetic signals during process portion 722. In process portion 711, the process can include directing the patient to undergo a second adjunctive therapy for a second period following the first period, with at least one characteristic of the second adjunctive therapy being different than the first. Accordingly, the process can include an overall treatment regimen that in turn includes both electromagnetic therapy and one or multiple adjunctive therapies. Aspects of the process 700 shown in FIG. 7 can be, but need not be, combined with aspects of the process 600 shown in FIG. 6. For example, in some embodiments, the first and second adjunctive therapy time periods (FIG. 7) can coincide with the first and second electromagnetic stimulation mode time periods (FIG. 6)—in other embodiments, these two sets of time periods can be independent of each other.

As described above, the adjunctive therapy can include one or more therapy types that are different than the electromagnetic signals applied as part of process portion 722. For example, the adjunctive therapy can include a systematized, directed behavioral activity, including a physical, cognitive, and/or psychiatric activity coordinated and possibly observed by a therapist. In terms of physical therapy, such activities can include grasping and releasing objects, stacking objects, placing objects in a box, manipulating objects, or other tasks that form part of a systematized physical therapy regimen. In at least some cases, these activities can form part of a standardized testing regimen as well, e.g., a Fugl-Meyer test.

The nature of the task can be selected depending upon the particular condition(s) the patient is suffering from. For example, if the patient is suffering from aphasia or another language-related disorder, the therapy task can be language-based and can include performing, attempting to perform, imagining patient performance of, and/or observing or noticing others perform any of a number of attempted speaking, listening, writing, and/or reading tasks. In some embodiments, the patient need not actually vocalize to successfully perform a task. Instead, the patient can be directed to merely think of a word, letter, phrase or other language component; or listen to or watch another individual perform the task. For example, the patient can be directed to silently generate a verb associated with a common noun, silently repeat a noun, silently retrieve a word based on a letter cue, or silently retrieve a word based on a visual cue. In particular cases, the patient can be directed to think of words beginning with the letter “c”, for example, or can be shown a picture of a cat and asked to think of the word represented by the picture. The patient can also be asked to respond non-verbally to an oral task that requires the patient to understand the difference between two auditory commands.

In other embodiments, the therapy activity can include a visual activity, auditory activity, gustatory activity, olfactory activity and/or haptic activity (e.g. pertaining to the sense of touch), again, depending upon the patient's specific disorder and/or symptoms. In some embodiments, an activity may comprise an observation activity. In general, an observation activity involves the patient observing or paying attention to one or more individuals who are performing particular activities or tasks or participating in or simulating particular behaviors (e.g., behaviors relating to movement, sensation, language, cognition, or emotion). In addition to actual performance or attempted performance of an activity or task, an observation activity may activate mirror neurons that are relevant to developing or restoring one or more types of functional abilities.

An observation activity may occur through real time or non-real time interaction (e.g., an audio/visual lesson or presentation) involving actual or simulated situations. Simulated situations may include patient observation of or interaction with another individual, a representation of another individual, or possibly a representation of the patient (e.g., using virtual reality). An observation activity may occur under the direction of or in response to instructions or suggestions received from a clinician or other individual; or in some instances an observation activity may be self-directed. Patient observation of others may further involve patient imagination of successful activity performance, or patient imitation of observed behaviors.

The adjunctive treatment need not be a systematized, directed physical therapy activity. For example, the adjunctive treatment can include activities of daily living (ADL). In other words, the patient can effectively perform adjunctive therapy by simply engaging in normal daily activities that might include getting dressed, eating, walking, talking and/or other activities. In still further embodiments, the adjunctive therapy need not include a behavioral therapy. For example, the adjunctive therapy can include a chemical substance or drug therapy. In any of these embodiments, the manner in which the adjunctive therapy is conducted, the type of adjunctive therapy undergone, and/or the presence or absence of any adjunctive therapy can be varied between the first time period and the second time period. In some embodiments, overall therapy provided during the first time period may be directed toward treating a first type of neurofunctional deficit or a first set of patient symptoms (e.g., hemiparesis), while the overall therapy provided during the second time period may be directed toward treating a second type of neurofunctional deficit or a second set of patient symptoms (e.g., aphasia). In other embodiments, the overall therapy provided to the patient during both time periods may be directed to a common deficit, but aspects of the overall therapy (e.g., the mode, signal delivery parameters, and/or adjunctive therapy) may differ from one time period to the next. The therapies provided during each time period may differ (e.g., due to different modes) while still being directed toward treatment of a common deficit.

For purposes of illustration, the variations in electromagnetic therapy parameters (e.g., mode) were described above independently of the variations in adjunctive therapy parameters. In practice, both parameters may be varied singly or in conjunction with each other in a wide variety of possible combinations. For example, the patient may undergo direct cortical stimulation via implanted electrodes, and may undergo directed physical therapy during a first time period. Both the electrical stimulation and the directed physical therapy may take place under the direct supervision of a trained practitioner. During the second time period, the patient may also receive direct cortical stimulation from the same or a different set of implanted electrodes, but may apply the stimulation by him or herself, or may have the stimulation triggered automatically without the direct involvement of a practitioner, or may have the stimulation provided in accordance with another mode. The adjunctive therapy during this second time period may shift from directed physical therapy to activities of daily living or other activities. For example, the patient may be coupled to a system that responds to feedback from the patient by automatically applying electromagnetic stimulation to the patient. If the adjunctive therapy is a physical activity (e.g., riding a stationary bike), the system can automatically detect the onset of the adjunctive therapy by detecting rotation of the bike wheels, and can automatically initiate or adjust electromagnetic stimulation by activating implanted electrodes via a wireless link. If the adjunctive therapy is a cognitive activity (e.g., responding to computer-based questions), the system can detect initiation of the adjunctive therapy by detecting an answer to a question, and can automatically initiate or adjust electromagnetic stimulation via the wireless link.

In another embodiments, the patient may receive practitioner-assisted electromagnetic therapy (e.g., via TMS or tDCS) during one period of time, and automated electromagnetic therapy in accordance with another mode (e.g., via an implanted electrode) during another period of time. In any of these embodiments, the manner in which the treatment is carried out (e.g., the mode, signal parameters and/or adjunctive therapy) is typically different when the treatment is directly supervised by a practitioner than it is when the treatment is not. This arrangement can allow the practitioner to directly supervise only those activities corresponding to particular treatment portions, while other (different) treatment portions can be carried out autonomously by a corresponding signal delivery system, or semiautonomously by the system with input from the patient.

### 3. Potential Results

One feature of many of the foregoing embodiments is that the manner(s) in which the electromagnetic therapy and/or the adjunctive therapy are conducted can be varied within and/or from one time period to another. One advantage of this feature is that it can reduce the likelihood for the patient's body to adapt or habituate to a particular type of electromagnetic and/or adjunctive therapy. As a result, the patient's neural system may be more likely to respond favorably to the therapy because the therapy varies. Another potential advantage associated with this feature is that it may improve the longevity of the effect achieved by the therapy. For example, it has been observed in some cases that a long-lasting effect of a combined electromagnetic/adjunctive therapy regimen completed during only a first period may tend to fall off somewhat over time. Accordingly, the second period of time may “boost” the effect achieved during the first period of time, and/or at least partially preserve the effects obtained during the first period of time. As a result, stimulation during the second period of time can enhance and/or increase the duration of the effects created during the first period of time. These effects can last for a period of at least days or weeks and in many cases, months or years, even though the treatment regimen (e.g., a series of treatment sessions over one, two or more periods of time) may take significantly less time.

Another feature of at least some of the foregoing embodiments is that they can produce a reduction in power consumed by one or more stimulation systems. This result can be achieved by combining modes, changing modes, and/or changing aspects of a particular mode. For example, switching from an implant mode to a nonimplant mode can effectively extend the life of an implanted power source. In another example, in certain situations switching from deep brain stimulation to cortical stimulation may result in a power savings, compared with using deep brain stimulation exclusively. If an implanted power source is non-rechargeable, combining modes, changing modes, and/or changing aspects of a mode may extend a power source lifetime (e.g., by 10%-50% or more) to a sufficient extent that the frequency of power source replacement surgeries may be decreased (e.g., by a commensurate or corresponding extent). Furthermore, combining or changing modes or altering mode aspects may eliminate the need for a power source replacement surgery following the use of a first implanted mode if the patient may be successfully treated using a second or subsequent non-implanted mode.

Still another feature of at least some of the foregoing embodiments is that the use of multiple modes (and/or multiple aspects of a particular mode) can synergistically enhance neural stimulation efficacy and/or address multiple symptoms and/or types of dysfunction. For example, deep brain stimulation may alleviate only some Parkinsonian symptoms, while cortical stimulation may relieve others (e.g., cognitive or affective symptoms). As another example, vagal nerve stimulation, TMS, and/or tDCS may treat an affective disorder such as depression or PTSD, while implanted cortical stimulation may (a) enhance such treatment, (b) facilitate the restoration or development of neural function associated with an affective or other disorder, or (c) treat another type of neurologic dysfunction from which the patient suffers (e.g., a pain syndrome). Similarly, peripheral stimulation can be used to address different symptoms than does CNS stimulation.

### C. Systems for Applying Electromagnetic Stimulation

FIGS. 8A-18 illustrate representative systems and devices for applying electromagnetic signals in accordance with the modes and signal delivery parameters described above. FIGS. 8A and 8B are isometric and cross-sectional views, respectively, of a signal delivery system 860 having a signal delivery device 850 configured to provide signals to a region of the cortex proximate to the pial surface. The signal delivery device 850 refers generally to the “end” portion of the system that delivers signals to the target neural population. For example, the signal delivery device 850 can include first and second electrodes 851 (identified individually by reference numbers 851a and 851b), and can be integrated with a signal source 874 (shown schematically), all of which are carried by a support member 852. The signal delivery device 850 can be electrically coupled to the signal source 874. The support member 852 can be configured to be implanted into the skull 544 or another intracranial region of a patient. For example, the support member 852 can include a housing 854 and an attachment element 855 connected to the housing 854. The housing 854 can be a molded casing formed from a biocompatible material that has an interior cavity for carrying the signal source 874.

Referring now to FIG. 8B, the signal delivery device 850 is implanted into the patient by forming an opening in the scalp 838 and cutting a hole 839 through the skull 544 and through the dura mater 840. The hole 839 should be sized to receive the housing 854, and in most applications, the hole 839 should be smaller than the attachment element 855. A practitioner inserts the support member 852 into the hole 839 and then secures the attachment element 855 to the skull 844. The attachment element 855 can be secured to the skull 844 using a plurality of fasteners 846 (e.g., screws, spikes, etc.) or an adhesive. Once implanted, the electrodes 851a, 851b contact and/or optionally press against a desired portion of the brain at the stimulation site. For example, the electrodes 851a, 851b can contact and press against the pia mater 841 surrounding the cortex 842.

FIGS. 8C and 8D schematically illustrate the signal delivery system 860, a portion of which is implanted in the cranium. Referring to FIG. 8C, the signal source 874 can include a power supply 861, a controller 862, a pulse generator 869, and a pulse transmitter 868. The power supply 861 can be a primary battery, such as a rechargeable battery or another suitable device for storing electrical energy. In other embodiments, the power supply 861 can be an RF transducer or a magnetic transducer that receives broadcast energy emitted from an external power source and converts the broadcast energy into power for the electrical components of the stimulation system 860. The controller 862 can include one or more computer-readable media having instructions for delivering command signals that effectuate neural stimulation. In an embodiment shown in FIGS. 8C and 8D, the controller 862 includes a wireless implanted portion 865 that responds to command signals sent by an external portion 864. The implanted portion 865, for example, can communicate with the external unit 864 by RF or magnetic links 875. The implanted portion 865 provides control signals to the pulse generator 869 in response to the command signals sent by the external portion 864. The pulse generator 869 can have a plurality of channels that send appropriate electrical pulses to the pulse transmitter 868, which is coupled to the electrodes 851. Suitable components for the power supply 861, the controller 862, the pulse generator 869, and the pulse transmitter 868 are known to persons skilled in the art of implantable medical devices.

Referring to FIG. 8D, those portions of the system 860 located within the housing 854 and carried by the support member 852 can be implanted in the manner described above with reference to FIGS. 8A and 8B. The external portion 864 can be located externally to the patient 536 so that the external portion 864 can be used to control the implanted portion 865. In one embodiment, several patients that require a common treatment can be simultaneously treated using a single external portion 864 by positioning the patients within the operational range of the external portion 864. In another embodiment, the external portion 864 can contain a plurality of operating codes and the implanted portion 865 for a particular patient can have an individual operating code. A single external portion or unit 864 can thus be used to treat a plurality of different patients by entering the appropriate operating code into the external portion 864 corresponding to the particular operating codes of the implanted portions 865 for the patients.

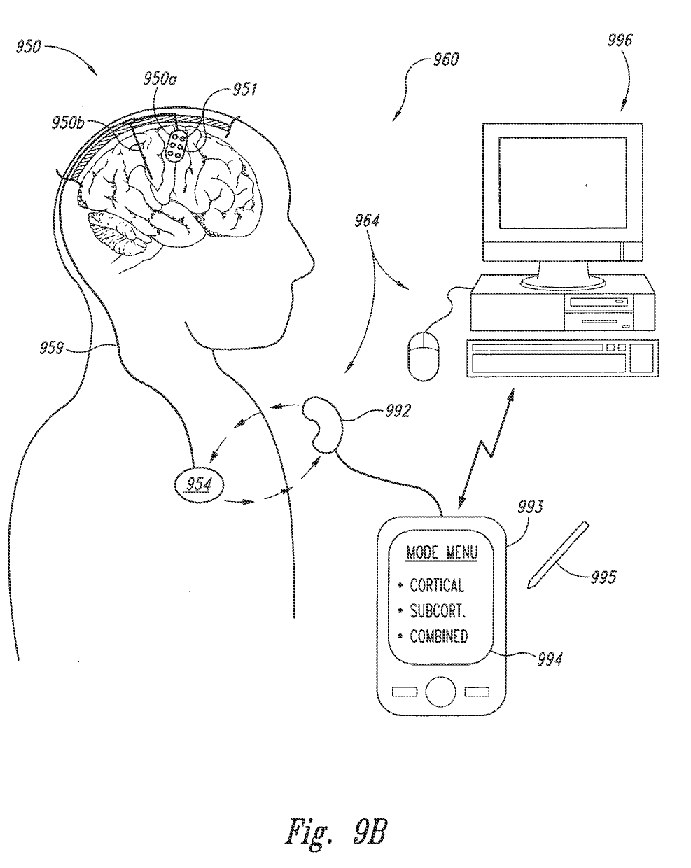

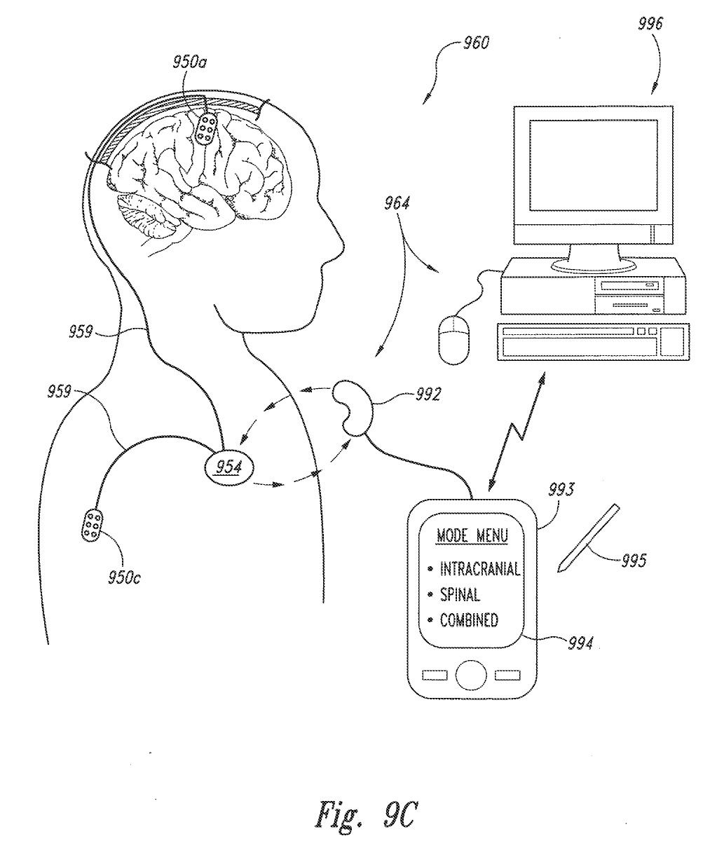

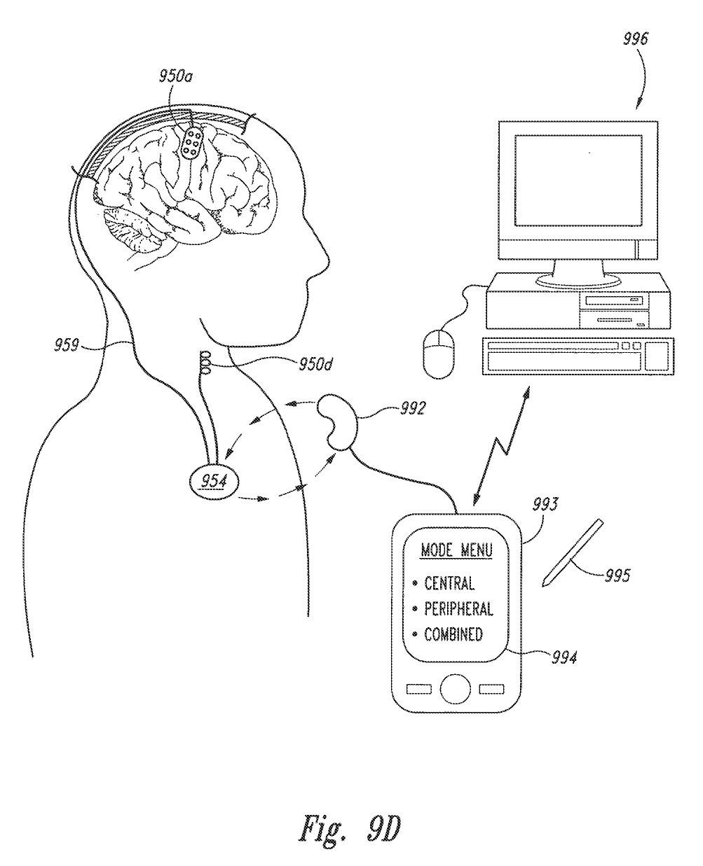

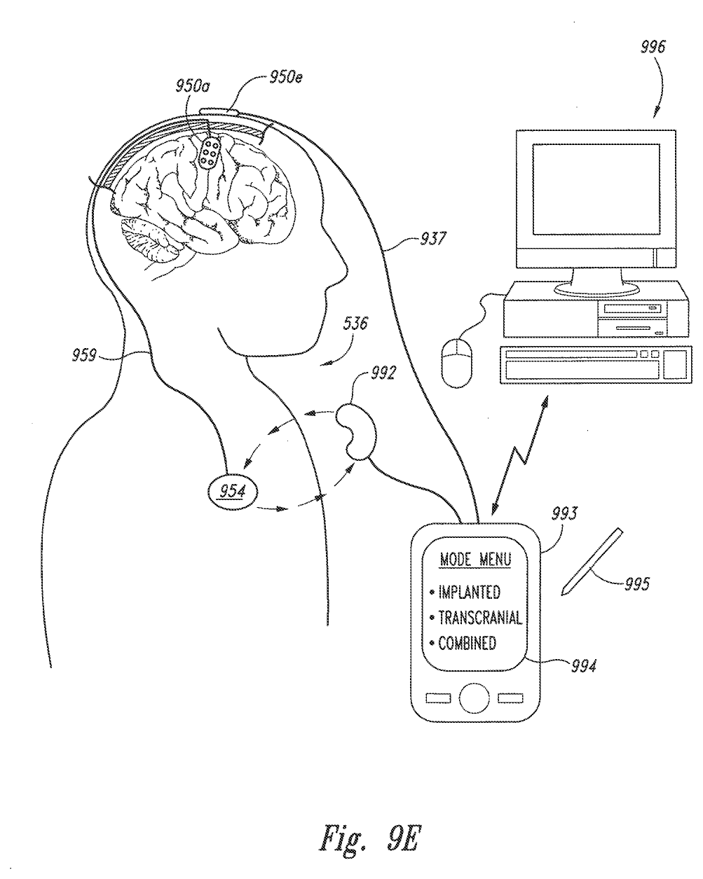

FIG. 9A illustrates a system 960 for applying electromagnetic stimulation to a patient via multiple modes in accordance with an embodiment to the invention. Each mode can include signal delivery by one or more signal delivery devices (e.g., cortical or subcortical electrodes, a cerebellar stimulator, a deep brain stimulator, a spinal column stimulator, a cranial nerve stimulator, transcranial electrodes and/or a transcranial magnetic stimulator). Signals can be provided to the signal delivery devices in accordance with any of the signal parameters described above (e.g., waveform parameters and location parameters). In one aspect of this embodiment, the system 960 can include at least one signal supply 974 (e.g., a signal generator) that provides signals to one or more signal delivery devices 950 (shown as signal delivery devices 950a, 950b . . . 950n). The signal supply 974 can include a power supply 961 coupled to a controller 962. The controller 962 controls signals that are transmitted to the signal delivery devices 950 (and ultimately, the patient) via a transmitter 968.

In one aspect of this embodiment, the controller 962 can be operatively coupled to multiple signal delivery devices 950 in a sequential manner. Accordingly, the controller 962 can provide stimulation to one signal delivery device 950 at a time via a mode that is commensurate with the corresponding signal delivery device. In other embodiments, the controller 962 can be configured to transmit signals to the patient via multiple signal delivery devices 950 simultaneously. In any of these embodiments, the controller 962 can include a mode selector 967 via which a practitioner can select the mode of treatment applied to the patient. The practitioner can do so via a user interface 963 (e.g., a touch screen, knob, or other suitable device). The controller 962 can further include a limiter 966 that prevents inappropriate signals from being transmitted by the transmitter 968 when such signals are not consistent with the mode selected via the mode selector 967. For example, if a practitioner selects a mode that has associated with it a peak current or peak frequency value, the limiter 966 can prevent the transmitter 968 from transmitting signals that exceed those values. The mode selector 967 can be a hardware switch or a software switch, and the limiter 966 can also include a hardware or software switch.

In still a further aspect of this embodiment, the limiter 966 can prevent signals from being transmitted to a signal delivery device 950 when such signals are not appropriate for that signal delivery device. For example, the system 960 can include a facility (e.g., hardware and/or software) for identifying whether the signal delivery device 950 coupled to the transmitter 968 is a first signal delivery device 950a or a second signal delivery device 950b. If only certain types of signals (e.g., AC or DC) and/or a certain range of signal parameters (e.g., voltage, current, frequency) are appropriate for the first signal delivery device 950a, the limiter 966 can be configured to prevent inappropriate signals from being transmitted to the first signal delivery device 950a when the first signal delivery device 950a is coupled to the controller 962. In particular embodiments, each signal delivery device 950a, 950b . . . 950n can have an identifying code that is recognized by the controller 962 so that the controller can automatically permit only signals having the proper characteristics from being transmitted to a corresponding signal delivery device. For example, a signal typically applied to an implanted electrode may be a set of biphasic pulses, while a signal applied to a tDCS electrode may be a direct current signal. As another example, during a therapy period, the limiter 966 can automatically prevent the transmission of suprathreshold signals to one or more implanted electrodes, or limit the duration or number of suprathreshold signals applied to such electrodes. In particular embodiments, the system can include a hardware arrangement (e.g., differently shaped connection ports for different types of signal delivery devices, or radio frequency identification (RFID) devices, chips, or tags corresponding to different signal delivery devices) to identify the signal delivery devices. Appropriate software (e.g., similar to that used to identify printers and other peripheral devices attached to a personal computer) can be used in addition to or in lieu of the hardware arrangement.

Certain components of the signal supply 974 can be housed in an implanted unit and/or an external unit. For example, the controller 962 can include an implanted unit that autonomously controls the electrical signals without further action by a practitioner or other individual. Alternatively, the implanted unit can communicate with an external unit that provides instructions regarding the type of electromagnetic signals provided to the patient. A power supply 961 can also be housed in an internal and/or external unit, but need not necessarily be co-housed with the controller. Further aspects of systems that have the foregoing characteristics and include one or more types of signal delivery devices are described below with reference to FIGS. 9B-18.

FIG. 9B illustrates a system 960 that includes multiple signal delivery devices 950 that can operate in accordance with multiple modes. For example, the system 960 can include one or more implanted cortical electrode devices 950a (having one or multiple electrodes 951) and one or more implanted subcortical (e.g., DBS) devices 950b, each which may be coupled with one or more leads 959 to an implanted housing 954. While FIG. 9B illustrates cortical and subcortical stimulation modes, other embodiments may provide for additional or different modes.

The implanted housing 954 can communicate via wireless telemetry with an external telemetry device 992. The external telemetry device 992 can form a portion of an external controller 964 that transfers program, control, data, and/or other signals (e.g., power signals) to and/or from the patient. Accordingly, the external controller 964 can include a hand-held unit 993 having a display screen 994, one or more input devices (e.g., keys, buttons, and/or a stylus 995), a processing unit, and one or more computer readable media for storing program instructions and data. The external controller 964 may provide a set of graphical menus or selection interfaces that provide a graphical user interface (GUI) to the practitioner. A practitioner can select modes using the hand-held unit 993 and can receive feedback (e.g., an indication of available modes and selected modes) via the display screen 994. In a particular embodiment shown in FIG. 9B, the available modes include a “cortical” mode, a “subcortical” mode, and a “combined” mode. The selection of a given mode or mode combination may result in the presentation of additional menus and/or selection interfaces to the practitioner. The additional menus and/or interfaces may facilitate the selection and/or specification of stimulation parameters corresponding to one or more modes, where such parameters may include current or voltage levels, pulse or burst characteristics, pulse or burst modulation functions, or spatial and/or temporal activation times or patterns associated with signals directed toward particular stimulation devices. The hand-held unit 993 can optionally communicate with an additional computer 996 (e.g., a desktop or other computer). Each of these modes can correspond to a type of CNS implant mode, described above with reference to FIG. 6.

The combination of cortical stimulation and deep brain stimulation may provide particular advantages to the patient in at least some embodiments. For example, deep brain stimulation can be used to “drive” or otherwise affect the excitability of a neural population within or proximate to the basal ganglia. The signals transmitted by the deep brain neural population can in turn affect neural populations at the cortex via neural projections, tracts and/or other neural signaling pathways. The response by the cortical neural population can be enhanced or modulated by the addition of the cortical stimulation, and the cortical neural population's response may in turn affect a deep brain population. In particular embodiments, the electromagnetic signals provided to a cortical neural population by the system 960 can have a selected temporal relationship to the electromagnetic signals provided to the deep brain population by the system 960. For example, the system 960 can stimulate the deep brain population and then follow up with stimulation to the cortical population at or close to the time signals generated by the deep brain population may be expected to affect the cortical population. In other embodiments, the two types of electromagnetic signals can be simultaneous. In still further embodiments, the two types of signals can be varied in other manners, for example, five minutes of deep brain signals alternating (and in some cases, at least partially overlapping) with five or some other number of minutes of cortical signals; or generally continuous deep brain stimulation in association with theta-burst or aperiodic cortical stimulation.

In other cases, deep brain stimulation can be combined with cortical stimulation in other manners. For example, deep brain stimulation can provide the primary electromagnetic treatment for a patient suffering from Parkinson's Disease, and can be provided on a continuous, nearly continuous, or generally continuous basis (e.g., 24/7 or at least during typical waking hours). Cortical stimulation can be provided simultaneously with the deep brain stimulation (and/or during interstices in the deep brain stimulation) to (a) facilitate or effectuate neuroplastic changes, (b) develop functionality that compensates at least in part for one or more patient symptoms, and/or (c) improve neuropsychological, neuropsychiatric, sensory, and/or motor functionality. Accordingly, the cortical stimulation can be provided at subthreshold levels, possibly in association with an appropriate adjunctive therapy program. In some embodiments, the cortical stimulation may comprise suprathreshold pulses or bursts.

In the foregoing manner, the addition of cortical stimulation to a regimen that typically employs deep brain stimulation may enhance patient functionality, in some instances at least in part because signaling changes associated with a cortical neural population may over time at least partially compensate for neurologic dysfunction associated with a deep brain population. In other cases, the reverse may apply, e.g., deep brain stimulation may enhance/expand upon an increase in functionality attainable from cortical stimulation alone.

In another aspect of an embodiment shown in FIG. 9B, the signal delivery devices can also be used to sense or receive signals. For example, particular electrodes 951 of the cortical stimulation device 950a can be used to detect electrocorticographic (ECOG) signals. ECOG signals may be used to characterize the patient's neurofunctional state, and may correspond to patient responses to cortical and/or deep brain stimulation. This response can be used as the basis for adjusting signal delivery parameters and/or changing signal delivery modes. As another example, a deep brain electrode may be used to sense neural activity to determine whether cortical stimulation is providing a given effect.

FIG. 9C illustrates the system 960 configured in accordance with another embodiment, in which the subcortical electrode 950b (FIG. 9B) is replaced with a spinal stimulation device 950c. Accordingly, the practitioner can select from an “intracranial” mode in which electromagnetic signals are delivered from the implanted cortical electrode device 950a, and a “spinal” mode in which electromagnetic signals are delivered from the spinal stimulation device 950c. The practitioner can also select a combined mode in which signals are provided by both devices. Each of these modes can correspond to a type of CNS implant mode, described above with reference to FIG. 6. Suitable spinal stimulation devices are available from Medtronic, Inc. of Minneapolis, Minn.

Plasticity may occur at several levels following spinal cord injury, including plasticity involving the cerebral cortex, brain stem, spinal cord, and peripheral nervous system. By providing electromagnetic signals to particular neuroanatomical sites associated with neuroplasticity, either individually or in combination, overall neuroplasticity may increase and/or be enhanced and therefore may facilitate the patient's recovery from a spinal cord injury. Appropriate stimulation sites may be identified in one or more manners described above, for example, through a neurofunctional localization procedure involving EEG or fMRI to characterize or identify particular types of neural activity (e.g., neural activity associated with neurofunctional change or recovery following neurologic damage), and/or a neurostructural identification procedure such as DTI to locate particular neural tracts or projections (e.g., neural tracts that remain viable following such damage, and which may be expected to successfully carry neural signals to facilitate or effectuate neuroplastic change).

FIG. 9D illustrates an embodiment of the system 960 configured to provide electromagnetic signals to a peripheral neural population in accordance with another embodiment of the invention. Accordingly, in a particular aspect of this embodiment, the system 960 includes a peripheral signal delivery device 950d. The peripheral signal delivery device 950d can be configured to stimulate one or more cranial nerves such as the vagus nerve (as shown in FIG. 9D), and/or other peripheral nerves. In an aspect of an embodiment shown in FIG. 9D, the peripheral signal delivery device is shown in combination with an implanted cortical electrode device 950a. In other embodiments, the peripheral signal delivery device 950d can be used in combination with other devices in accordance with other modes. Signals can be provided to the peripheral signal delivery device 950d in combination with, or separately from signals provided to the implanted cortical electrode device 950a, as indicated by the “intracranial,” “peripheral,” and “combined” modes identified at the display screen 994. In this case, the intracranial mode represents a type of CNS implant mode, and the peripheral mode represents a type of peripheral implant mode.

The combination of cortical stimulation and cranial (e.g., vagal) and/or other peripheral nerve stimulation may enhance neural stimulation efficacy beyond that of either of such modes individually. Vagal nerve stimulation may affect cerebral blood flow or alter neural activity in various cortical and/or subcortical regions, including the orbitofrontal cortex, the somatosensory cortex, the insular cortices, the thalamus, the hypothalamus, the amygdala, the cingluate gyrus, and other regions (Jeong-Ho Chae et al., “A review of the new minimally invasive brain stimulation techniques in psychiatry,” Rev. Bras. Psiquiatr., Vol. 23 No. 2, Sao Paulo, June 2001). Accordingly, the combination of cortical stimulation and cranial nerve stimulation (e.g., in a sequential, partially overlapping, or simultaneous manner) may aid the establishment or maintenance of a desired neural outcome (e.g., a metabolic shift away from a hypometabolic or hypermetabolic state; or a modulation of a maladaptive neuroplastic condition). The combination of cortical stimulation and cranial nerve stimulation, possibly in association with one or more adjunctive therapies, may alternatively or additionally enhance the restoration and/or development of neural function (e.g., in patients suffering from neurologic damage or other neurologic dysfunction).

The identification of particular brain regions that exhibit acute or chronic changes in neural activity or neural metabolite levels as a result of cranial or other peripheral nerve stimulation may aid in (a) identifying one or more sites at which to implant cortical electrodes, (b) determining particular cortical regions to which stimulation signals should be directed across different time periods, (c) establishing or adjusting cortical and/or peripheral stimulation parameters (e.g., current or voltage levels, signal polarity), or (d) establishing or adjusting one or more adjunctive therapies. Such brain regions may be identified, for example, using a neurofunctional localization procedure (e.g., fMRI) to measure neural activity levels before, during, and/or after one or more cranial nerve stimulation periods, either independent of or in conjunction with patient performance or attempted performance of one or more relevant neurofunctional activities or tasks.

The combination of cortical stimulation and vagal or other cranial nerve stimulation may reduce certain symptoms associated with neuropsychiatric disorders (e.g., depression or anxiety), movement disorders, auditory disorders (e.g., tinnitus or auditory hallucinations), or other conditions. The benefits that may be achieved with the combination of cortical stimulation and cranial nerve stimulation may be similar or analogous to those achieved with deep brain stimulation alone or the combination of deep brain stimulation and cortical stimulation. Because both cortical stimulation and vagal stimulation are each significantly less invasive than deep brain stimulation, their combination may provide a favorable alternative to deep brain stimulation alone or deep brain stimulation in combination with cortical stimulation.

FIG. 9E illustrates an embodiment of the system 960 configured to provide electromagnetic signals via tDCS. Accordingly, the system 960 can include a set of tDCS signal delivery devices 950e, in combination with one or more other signal delivery devices, such as the implanted cortical electrode device 950a shown in FIG. 9E. In general, the set of tDCS signal delivery devices 950e includes a stimulating or source electrode as well as a return or circuit completion electrode, in a manner understood by those skilled in the art. As discussed above, the practitioner can elect to provide electromagnetic stimulation via one or more modes by entering the appropriate instructions at the handheld unit 993. The modes shown in FIG. 9E include an “implanted” mode (e.g., a type of CNS implant mode) and a “transcranial” mode (e.g., a type of CNS non-implant mode). The practitioner can also use the handheld unit 993 (and/or another input device) to define signal delivery parameters. The signal delivery parameters can include the waveform parameters (e.g., current, voltage, frequency and others) described above and, in some cases, can also include a specification of one or more locations to which particular electromagnetic signals are directed (e.g., tDCS signals may be directed to a healthy hemisphere in association or conjunction with implanted cortical stimulation signals directed to an impaired hemisphere, or vice-versa). When an implanted cortical electrode device 950a includes multiple electrodes 951, defining the signal delivery parameters can include defining which electrodes 951 transmit signals, as well as the type of signal transmitted by each electrode 951.

In other embodiments, other combinations of signal delivery devices are possible. For example, such combinations can include the combination of a transcranial magnetic stimulation device with a transcranial direct current stimulation device. The selection of a particular system and/or signal delivery device can be based at least in part on the type, extent, or severity of the patient's neurologic dysfunction, and/or the patient's amenability to particular signal delivery devices.

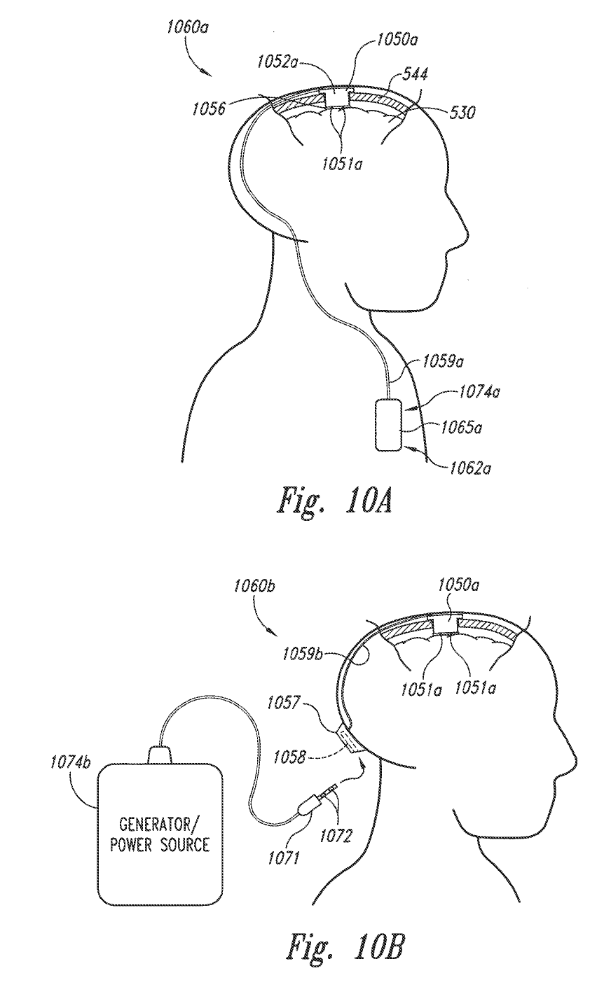

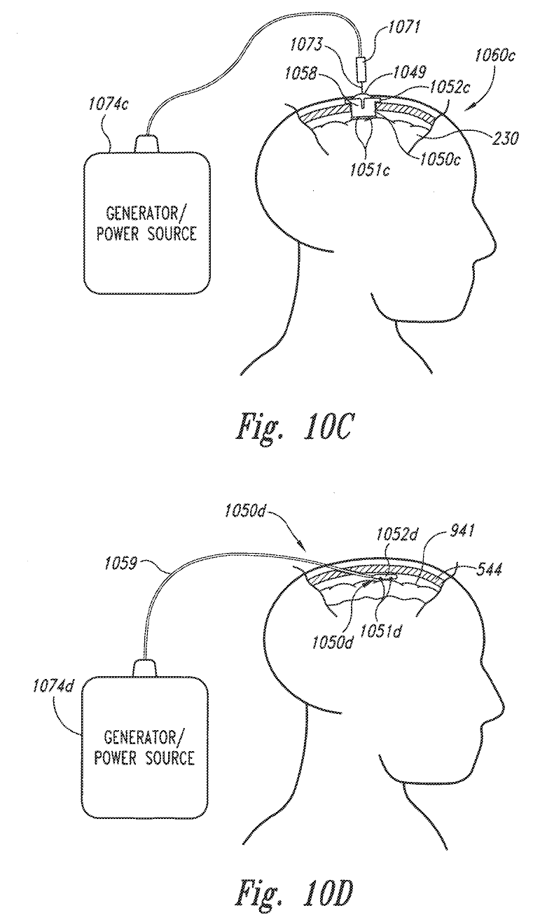

FIG. 10A is a schematic illustration of a system 1060a having a signal source 1074a that includes components located remotely from a corresponding signal delivery device 1050a. The signal delivery device 1050a can include a support member 1052a carrying a plurality of electrodes 1051a. The support member 1052a can include a forcing element 1056 that urges the electrodes 1051 into contact with the brain 530. The signal source 1074a can include components described above with reference to FIGS. 8A-8D, but is not “integrated” because it is not carried by the support member 1052a. The signal source 1074a can be coupled to the electrodes 1051a by a cable 1059a. In a typical application, the cable 1059a is implanted subcutaneously in a tunnel from a subclavicular region, along the back of the neck, and around the skull. The signal source 1074a can include a controller 1062a with an internal portion 1065a that operates either autonomously or in cooperation with an external portion in a manner generally similar to that described above with reference to FIGS. 8C-8D.

FIG. 10B is a schematic cross-sectional view of a system 1060b having a signal source 1074b coupled to the signal delivery device 1050a in accordance with another embodiment of the invention. The signal delivery device 1050a can be coupled to an external receptacle 1057 having an electrical socket 1058. An implanted lead line 1059b couples the electrodes 1051a to contacts (not shown) in the socket 1058. The lead line 1059b can be implanted in a subcutaneous tunnel or other passageway in a manner known to a person skilled in the relevant art. The signal delivery device 1050a, however, does not have an internal pulse system carried by the portion of the device that is implanted in the skull 537. Instead, the signal source 1074b is positioned external to the patient and transmits signals to the implanted signal delivery device 1050a via the external receptacle 1057. Accordingly, the signal source 1074b can have an electrical connector 1071 with a plurality of contacts 1072 configured to engage the contacts within the receptacle 1057. The signal source 1074b can also have a power supply, controller, pulse generator, and pulse transmitter to generate the electrical pulses. In operation, the signal source sends electrical pulses to the signal delivery device 1050b via the connector 1071, the receptacle 1057, and the lead line 1059b.

FIG. 10C illustrates a system 1060c having an external signal source 1074c that communicates with an implanted signal delivery device 1050c in accordance with another embodiment of the invention. The signal delivery device 1050c can include a support structure 1052c having a socket 1058, a plurality of contacts arranged in the socket 1058, and a diaphragm 1049 covering the socket 1058. The signal delivery device 1050c can also include a forcing element and a plurality of electrodes 1051c attached to the forcing element to urge the electrodes 1051 into contact with the brain 230. In another embodiment, the forcing element can be eliminated. In either embodiment, each electrode 1051 is directly coupled to one of the contacts within the support structure 1052c.

The signal delivery device 1050c receives electrical pulses from the external signal source 1074c, which can in turn include a power supply, controller, pulse generator, and pulse transmitter. The external signal source 1074c can also include a plug 1071 having a needle 1073 and a plurality of contacts arranged on the needle to contact the internal contacts in the socket 1058. In operation, the needle 1073 is inserted into the socket 1058 to engage the contacts on the needle with the contacts on the socket, and then the signal source 1074c is activated to transmit electrical pulses to the electrodes 1051.

FIG. 10D is a schematic cross-sectional view of an implantable signal delivery device 1050d configured in accordance with another embodiment of the invention. In one embodiment, the signal delivery device 1050d has a support structure 1052d and a plurality of electrodes 1051d coupled to the support structure 1052d. The support structure 1051d can be configured to be implanted under the skull 544 between an interior surface of the skull 544 and the pial surface of the brain. The support structure 1052d can be a flexible or compressible body such that the electrodes 1051d contact the pia mater 841 when the signal delivery device 1050d is implanted under the skull 544. In other embodiments, the support structure 1052d can position the electrodes 1051d so that they are proximate to, but not touching, the pia mater 841.

The signal delivery device 1050d can receive electrical pulses from an external signal source 1074d. For example, the external signal source 1074d can be electrically coupled to the signal delivery device 1050d by a lead line 1059 that passes through a hole 1039 in the skull 544. In another embodiment, the signal delivery device 1050d can be coupled to an integrated pulse system and external control portion generally similar to the pulse systems and control portions described above with reference to FIGS. 8A-8D.

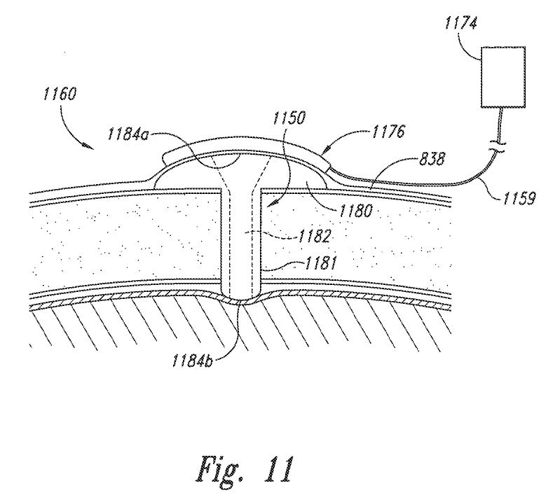

FIG. 11 illustrates an intracranial electrode system 1160 configured in accordance with an embodiment of the invention. The electrode system 1160 can include an electrical energy transfer device (ETD) 1176 externally placed adjacent to a patient's scalp 838 to couple electrical energy from a signal source 1174 to an intracranial electrode signal delivery device 1150. A lead wire 1159 may couple the ETD 1176 to the signal source 1174. The signal source may be of an identical, essentially identical, analogous, or different type relative to the signal generators shown in FIGS. 10B-10D.

The ETD 1176 can include a conventional adhesive patch electrode commonly used for providing an electrical coupling to a particular location on a patient. The signal delivery device 1150 can include a head 1180 coupled to a shaft 1181. The head 1180 and shaft 1181 may be integrally formed of an electrically conductive material forming a conductive core 1182 that forms an electrical energy conduit. The conductive core 1182 may extend throughout a portion or along the entire length of the signal delivery device 1150. The conductive core 1182 may be carried by or encased in an electrically insulating material or cladding 1183. The conductive core 1182 may extend from an upper or proximal contact surface 1184a to a lower or distal contact surface 1184b. Contact surfaces 1184a and 1184b provide a signal exchange interface of the conductive core 1182. In one embodiment, the signal delivery device 1150 includes a distal contact surface 1184b that operates as a single electrode, and which may be positioned epidurally or subdurally. In other embodiments, the signal delivery device 1150 can include multiple contacts or electrode elements that may be coupled to a single potential or power channel, or to individual potentials or power channels. An electromagnetic signal return path may be provided by one or more additional signal delivery devices 1150 (which may be positioned proximate to or remote from a stimulation site), and/or another ETD 1176 in a manner understood by those skilled in the art. The ETD 1176 can include an energy transfer patch 1185 that may have several layers. In general, an ETD 1176 can include an outer flexible, insulating, and/or articulated layer 1186, an electrically conductive layer 1187, and a gel layer 1188. The conductive layer 1187 may include a conductive material (e.g., aluminum) for carrying or conveying an electrical signal. The conductive layer 814 may be appropriately shaped (e.g., oval or elliptical) for conforming to a portion of the skull's rounded surface.

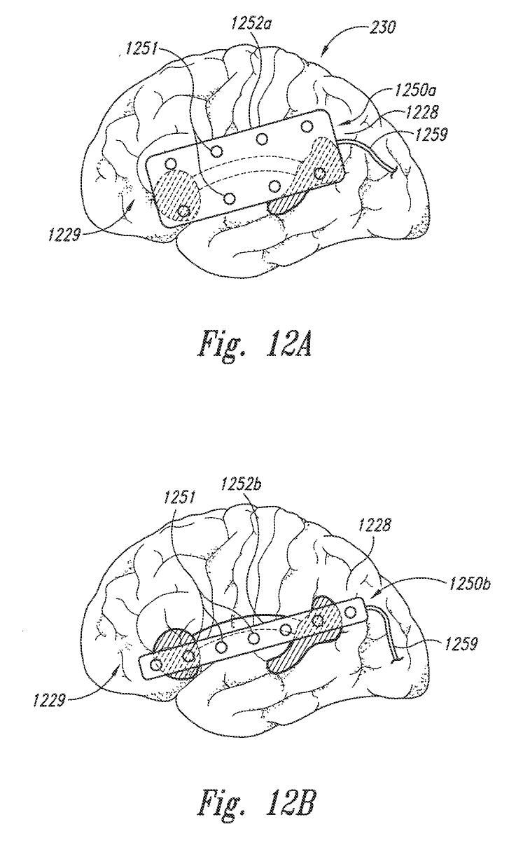

FIG. 12A is an isometric illustration of the brain 230 with a signal delivery device 1250a positioned to provide stimulation in accordance with another embodiment of the invention. In one aspect of this embodiment, the signal delivery device 1250a includes a support 1252a carrying a plurality of electrodes 1251 (eight are shown in FIG. 12A). In a further aspect of this embodiment, the signal delivery device 1250a is positioned to cover a plurality of cortical regions that may be associated with a particular patient condition and/or treatment regimen. For example, the signal delivery device 1250a can be configured to extend over the cortical areas responsible for carrying out language-based tasks when the patient suffers from a language-related disorder. Accordingly, in one embodiment, the signal delivery device can be sized to extend generally from the inferior frontal lobe 1229 to the inferior parietal lobe 1228, and can include electrodes 1251 located to stimulate any of a plurality of areas between and adjacent to these structures. In any of these embodiments, the signal delivery device 1250a can also include a lead 1259 coupled to a signal source.

One feature of an embodiment of the signal delivery device 1250a described above with reference to FIG. 12A is that it can include an array of electrodes 1251 that are spaced apart from each other, for example, along two transverse axes. Accordingly, each electrode 1251 can be positioned to stimulate a particular region of the brain 230. An advantage of this arrangement is that a practitioner can stimulate multiple sites of the brain 230 (either simultaneously or sequentially) with a single signal delivery device 1250a. In one embodiment, the practitioner can stimulate multiple sites of the brain 230 (rather than a single site) to produce enhanced benefits for the patient. In another embodiment, the practitioner can use a signal delivery device 1250a having an array of electrodes 1251 when it is initially uncertain which area(s) of the patient's brain 230 should be stimulated to produce the most beneficial effect. Accordingly, a practitioner can stimulate a particular area of the brain 230 with one of the electrodes 1251, observe the effect on the patient, and if the effect is not the desired effect, stimulate another area of the brain 230 with another of the electrodes 1251 and observe the resulting effect, all with a single, implanted device 1250a. In still another embodiment, the practitioner can apply stimulation to different sites for different lengths of time, and/or the practitioner can independently vary other stimulation parameters applied to the electrodes 1251. For example, the practitioner can couple various pairs of the electrodes 1251 to operate in a bipolar manner, or the practitioner can provide a separate, remote electrode (not shown) and operate all the electrodes 1251 carried by the support in a monopolar manner.

In another embodiment shown in FIG. 12B, the practitioner can implant a generally strip-shaped signal delivery device 1250b in the patient. In one aspect of this embodiment, the signal delivery device 1250b can include an elongated support 1252b carrying a plurality of linearly aligned electrodes 1251 coupled to a lead 1259. The signal delivery device 1250b can be positioned to extend over a relatively narrow band between the inferior frontal lobe 1229 and the inferior parietal lobe 1228. In one aspect of this embodiment, the signal delivery device 1250b can include six electrodes 1251, and in other embodiments, the electrode assembly 1250b can include more or fewer electrodes 1251b. In any of these embodiments, the electrodes 1251b can be selectively activated, simultaneously or sequentially, to provide the patient with a therapeutically effective treatment.

In other embodiments, the signal delivery devices 1250a, 1250b can have arrangements other than those described above. For example, other signal delivery devices can have support members with shapes other than those shown in FIGS. 12A and 12B, including irregular shapes. In still further embodiments, the electrodes can be distributed over the support members in irregular patterns, for example, to align with sites at the brain 230 most likely to be selected for stimulation. The signal delivery devices can be positioned adjacent to the language centers of the brain, as described above, and/or proximate to other areas of the brain, depending on the patient's condition and disorder.

In one aspect of embodiments described above with reference to FIGS. 12A-12B, the signal delivery devices are positioned over the left hemisphere of the patient's brain because the language centers of the brain are typically concentrated there. In other embodiments, the signal delivery devices can be positioned on the right side of the patient's brain to stimulate right hemisphere neurons. Accordingly, the signal delivery device 1250b can be positioned adjacent to the brain structures homologous to those described above with reference to FIGS. 12A-12B. For example, the stimulation applied to the right side of the patient's brain 230 can recruit right-side neurons to take over functions normally provided by (now defective) tissue on the left side of the patient's brain 230. In either embodiment, it can be advantageous to have a plurality of electrodes to allow flexibility in treating the patient's disorder.

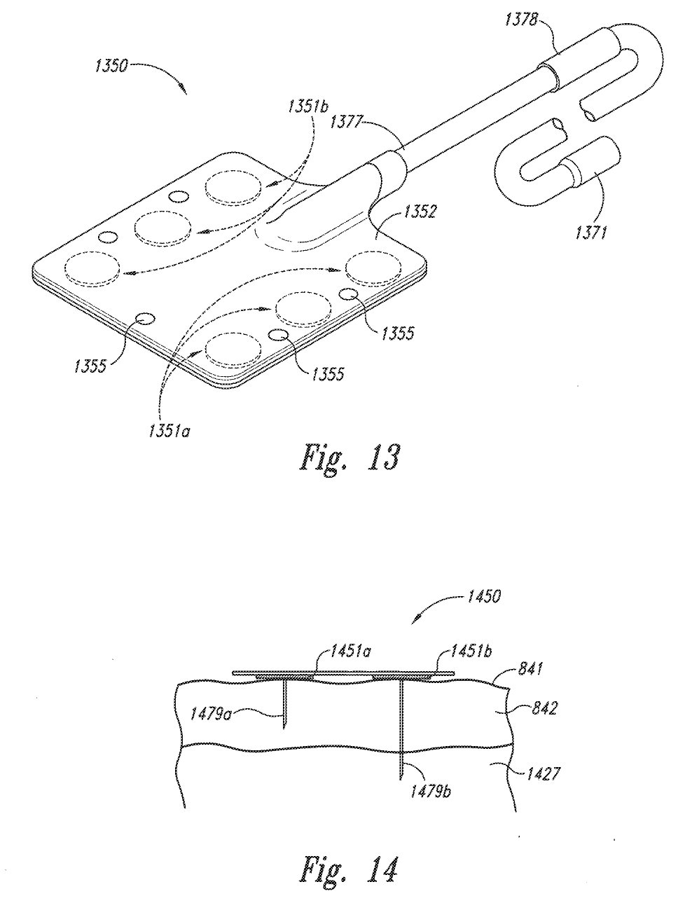

FIG. 13 is a top partially hidden isometric view of an implantable signal delivery device 1350 configured in accordance with an embodiment of the invention. In one aspect of this embodiment, the signal delivery device 1350 includes an electrode array comprising a first plurality of electrodes 1351a and a second plurality of electrodes 1351b (collectively referred to as electrodes 1351). The electrodes 1351 can be carried by a flexible support member 1352 configured to place each electrode 1351 in contact with a stimulation site of a patient when the support member 1352 is placed at the stimulation site. The electrodes 1351 are connected to conductors or lead lines (not shown in FIG. 13) housed in a cable 1377. A distal end of the cable 1377 can include a connector 1371 for connecting the lead lines to an implanted pulse generator (IPG) or other signal source. In operation, the first plurality of electrodes 1351a can be biased at a first potential and the second plurality of electrodes 1351b can be biased at a second potential at any given time. The different potentials can generate electrical pulses in the patient at, or at least proximate to, the stimulation site. In a different embodiment, all of the electrodes can be at the same potential for a unipolar stimulation process.

Although the signal delivery device 1350 of the illustrated embodiment includes a 2.times.3 electrode array (i.e., 2 rows of 3 electrodes each), in other embodiments, electrode assemblies in accordance with the present invention can include more or fewer electrodes in other types of symmetrical and asymmetrical arrays. For example, in one other embodiment, such a signal delivery device 1350 can include a 2.times.1 electrode array. In another embodiment, such a signal delivery device can include a 2.times.5 electrode array. In a further embodiment, such a signal delivery device can include a single electrode for unipolar stimulation.

The signal delivery device 1350 can include one or more coupling apertures 1355 extending through the periphery of the support member 1352. The coupling apertures 1355 can facilitate attachment of the signal delivery device to the dura mater at, or at least proximate to, a stimulation site. The signal delivery device 1350 can also include a protective sleeve 1378 disposed over a portion of the cable 1377 to protect the cable 1377 from abrasion resulting from contact with the edge of an access hole formed in the patient's skull.

FIG. 14 is a side elevational view of a signal delivery device 1450 configured in accordance with another embodiment of the invention. In this embodiment, the signal delivery device 1450 has multiple electrodes 1451, two of which are shown in FIG. 14 as a first electrode 1451a and second electrode 1451b. The electrodes 1451 also include first and second electrically conductive pins 1479a, 1479b. The pins 1479a, 1479b can be configured to extend below the pial surface of the cortex. For example, because the length of the first pin 1479a is less than the thickness of the cortex 842, the tip of the first pin 1479a will accordingly conduct the electrical pulses to a stimulation site within the cortex 842 below the pial surface. The length of the second pin 1479b is greater than the thickness of the cortex 842 to conduct the electrical pulses to a portion of the brain below the cortex 842, such as a deep brain region 1427. The lengths of the pins are selected to conduct the electrical pulses to stimulation sites below the pia mater 841. As such, the length of the pins 1479a, 1479b can be the same for each electrode or different for individual electrodes. Additionally, only a selected portion of the electrodes and the pins can have an exposed conductive area. For example, the electrodes 1451 and a portion of the pins 1479 can be covered with a dielectric material so that the only exposed conductive material is at the tips of the pins. It will also be appreciated that any of the electrode configurations described above can be configured to apply an electrical current to stimulation sites below the pia mater by providing pin-like electrodes in a matter similar to that shown in FIG. 14.

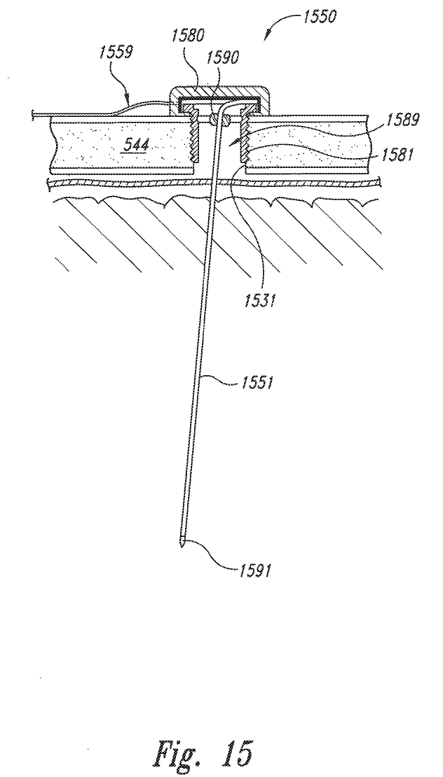

FIG. 15 schematically illustrates a subcortical or deep brain intracranial signal delivery device 1550 in accordance with another embodiment of the invention. This device 1550 includes a head 1580 having a threaded shaft 1581 with an axially-extending opening 1589 extending through the length of the head 1580. The head 1580 may also include a gimbal fitting 1590 configured to slidably receive a length of a conductive member 1551.

The gimbal fitting 1590 is configured to allow an operator greater control over the placement of an electrically conductive tip 1591 of the conductive member 1551. In use, the tip 1591 of the conductive member 1551 will be threaded through an opening in the gimbal fitting 1590. By pivoting the gimbal fitting 1590 with respect to the threaded shaft 1581, the angular orientation of the conductive member 1551 with respect to a pilot hole 1531 in the skull 544 can be accurately controlled. Once the operator determines that the conductive member 1551 is at the appropriate angle, e.g., using a surgical navigation system, the operator may advance the conductive member 1551 to position the conductive tip 1591 at a target site. Once the tip 1591 is in position, a capped lead 1559 may be press-fitted on the head 1580 of the device 1550. This will crimp the proximal length of the connective member 1551 between the head 1580 and the conductive inner surface of the cap, providing an effective electrical connection between the conductive member 1551 and the lead 1559. In other embodiments, the signal delivery device 1550 can have other configurations suitable for deep brain stimulation. Such devices are available from Medtronic, Inc. of Minneapolis, Minn.

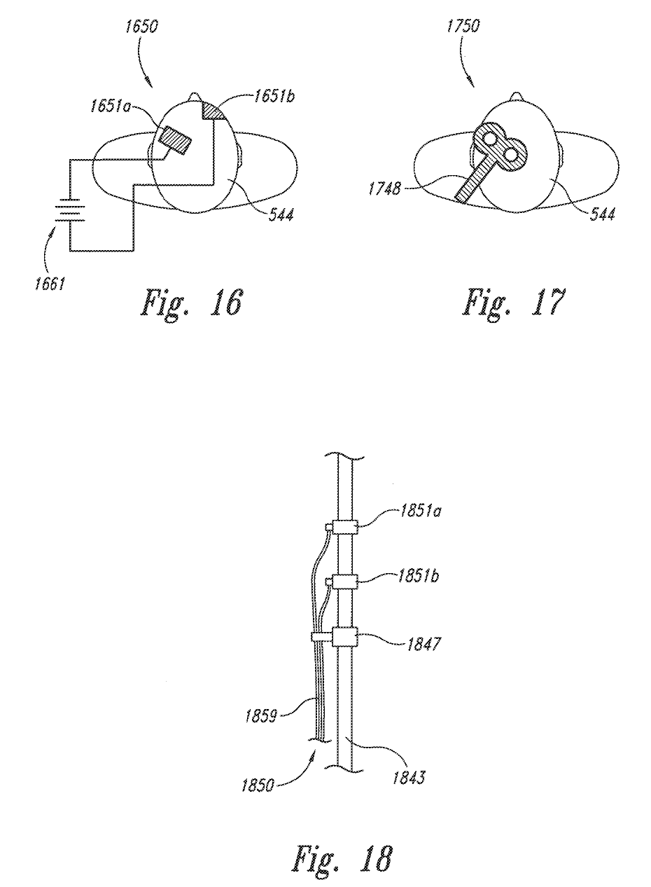

FIG. 16 illustrates a signal delivery device 1650 configured for transcranial direct current stimulation (tDCS) in accordance with still another embodiment of the invention. In one aspect of this embodiment, the entire signal delivery device 1650 can be positioned external to the patient's skull 544. The signal delivery device 1650 can include two electrodes 1651 (shown as a first electrode 1651a and a second electrode 1651b) that supply direct current through the patient's scalp and skull to the cortical tissue beneath. The electrodes 1651 are then coupled to a direct current power supply 1661.

FIG. 17 illustrates a signal delivery device 1750 configured to provide repetitive transcranial magnetic stimulation (rTMS) to the patient in accordance with still another embodiment to the invention. The signal delivery device 1750 can include a magnetic coil 1748 that is positioned over a target neural area so as to provide electromagnetic stimulation to the cortical tissue through the patient's scalp and skull 544. If the patient has previously had electrodes implanted beneath the skull, these electrodes may aid in conducting electromagnetic signals from the magnetic coil 1748 to the target neural tissue even though the electrodes are not directly applying electromagnetic signals in such an embodiment. Further aspects of both tDCS and rTMS techniques and systems are disclosed by Lang et al. in The Journal of Biological Psychiatry 2004; 56: 634-639, incorporated herein in its entirety by reference.

In still further embodiments, the electromagnetic stimulation may be applied to neural tissue other than cortical or deep brain tissue. For example, FIG. 18 illustrates a signal delivery device 1850 configured to provide electrical stimulation to the patient's vagal nerve 1843. The signal delivery device 1850 can include two electrodes 1851 (shown as a first electrode 1851a and a second electrode 1851b) that are each positioned adjacent to the vagal nerve 1843. The signal delivery device 1850 can further include an anchor tether 1847 that secures both the electrodes 1851 and a bundle of lead lines 1859 in position relative to the vagal nerve 1843. Suitable signal delivery devices for vagal nerve stimulation are available from Cyberonics, Inc. of Houston, Tex., under the trade name VNS Therapy. An advantage of providing stimulation to the vagal nerve or other cranial nerve is that this process need not include access through the patient's skull. This technique may also be less likely to impact non-targeted neural tissue because it may be easier to stimulate the cranial nerves at locations relatively distant from other neural tissue.

From the foregoing, it will be appreciated that specific embodiments of the invention have been described herein for purposes of illustration, but that various modifications may be made without deviating from the invention. For example, many of the techniques described above in the context of cortical stimulation from within the skull can also be applied to cranial nerves (e.g., the vagal nerve) that may be accessible without entry directly through the patient's skull. Many of the techniques described above in the context of subthreshold stimulation may be applied as well in the context of superthreshold stimulation. Aspects of the invention described in the context of two time periods may apply to more time periods (e.g., three or more) in other embodiments. Electromagnetic signals described in some embodiments as stimulation signals may be replaced with inhibitory signals in other embodiments, for example, by changing signal frequency and/or other signal delivery parameters. Aspects of the invention described in the context of particular embodiments may be combined or eliminated in other embodiments. For example, many of the signal delivery devices described above may have other configurations and/or capabilities in other embodiments. Several of those embodiments are described in the following pending U.S. Applications, all of which are incorporated herein by reference: Ser. No. 10/606,202, filed Jun. 24, 2003; Ser. No. 10/410,526, filed Apr. 8, 2003; Ser. No. 10/731,892, filed Dec. 9, 2003; Ser. No. 10/742,579, filed Dec. 18, 2003; and Ser. No. 10/891,834, filed Jul. 15, 2004. Further, while advantages associated with certain embodiments of the invention have been described in the context of those embodiments, other embodiments may also exhibit such advantages, and not all embodiments need necessarily exhibit such advantages to fall within the scope of the invention. Accordingly, the invention is not limited except as by the appended claims.

What is claimed:

1. A method for treating a neural condition, comprising:
directing an application of electromagnetic signals to a target neural population of a patient over a first period of time in accordance with a first mode; and
directing an application of electromagnetic signals to the patient over a second period of time different than the first period of time in accordance with a second mode different than the first mode, wherein each of the first and second modes is selected from among the following modes:
(a) a CNS implant mode in which electromagnetic signals are applied to the patient's central nervous system via at least one electrode implanted in the patient's body;
(b) a CNS non-implant mode in which electromagnetic signals are applied to the patient's central nervous system from a signal delivery device that is not implanted in the patient's body;
(c) a peripheral implant mode in which electromagnetic signals are applied to the patient's peripheral nervous system via at least one electrode implanted in the patient's body; and
(d) a peripheral non-implant mode in which electromagnetic signals are applied to the patient's peripheral nervous system from a signal delivery device that is not implanted in the patient's body.

2. The method of claim 1 wherein directing an application of electromagnetic signals over at least one of the first and second time periods is performed at least in part by a practitioner.

3. The method of claim 1 wherein directing an application of electromagnetic signals over at least one of the first and second time periods is performed at least in part by a computer-readable medium.

4. The method of claim 1 wherein applying electromagnetic stimulation in accordance with the CNS implant mode includes applying electromagnetic stimulation from at least one electrode implanted within the patient's skull.

5. The method of claim 1 wherein applying electromagnetic stimulation in accordance with the CNS non-implant mode includes applying electromagnetic stimulation from a location external to the patient's skull.

6. The method of claim 1 wherein applying electromagnetic stimulation over the first period of time includes applying electromagnetic stimulation from a location proximate to the cerebral cortex and within the patient's skull, and wherein applying electromagnetic stimulation over the second period of time includes applying transcranial direct current stimulation from a location external to the patient's skull.

7. The method of claim 1 wherein applying electromagnetic stimulation over the first period of time includes improving a brain function of the patient, and wherein applying electromagnetic stimulation over the second period of time includes preserving, extending or both preserving and extending the improvement in brain function obtained during the first period of time.

8. The method of claim 1 wherein applying electromagnetic stimulation over one or both of the periods of time includes improving a brain function of the patient for a period of days, weeks, months or years.

9. The method of claim 1, further comprising applying a conditioning stimulus within minutes or hours before or after applying the electromagnetic stimulation during at least one of the first and second periods of time.

10. The method of claim 1 wherein:
directing an application of electromagnetic signals in accordance with a first mode includes directing electromagnetic signals with a first set of signal parameters, including at least one of:
a first current level, a first voltage level, a first pulse width, a first representative frequency, a first modulation function, and a first polarity; and wherein
directing an application of electromagnetic signals in accordance with a second mode includes directing electromagnetic signals with a second set of signal parameters, including at least one of:
a second current level, a second voltage level, a second pulse width, a second representative frequency, a second modulation function, and a second polarity; and wherein
at least one aspect of the second set of signal parameters is different than a corresponding aspect of the first set of signal parameters.

11. The method of claim 1, further comprising directing an application of signals in accordance with the first mode to a first location of the patient's body, and directing an application of signals in accordance with the second mode to a second location of the patient's body, different than the first.

12. The method of claim 11 wherein the first and second locations are selected from among the following locations:
a location at the cortex, a location above the cortex, a location below the cortex, a location at least proximate to the vagal nerve, a cerebellar location, a location at least proximate to a peripheral nerve, and a location at least proximate to a muscle.

13. The method of claim 11 wherein the first location is at least proximate to the patient's cortex.

14. The method of claim 11 wherein the first and second locations are at the same hemisphere of the patient's brain.

15. The method of claim 11 wherein the first and second locations are at different hemispheres of the patient's brain.

16. The method of claim 11 wherein electromagnetic signals directed during the first time period are directed toward a first neural population and electromagnetic signals directed during the second time period are directed to a second neural population different than the first.

17. The method of claim 11 wherein electromagnetic signals applied during the first and second time periods are directed to the same neural populations.

18. The method of claim 1 wherein at least one of the first and second time periods has a duration of hours, days, or weeks.

19. The method of claim 1 wherein the first and second time periods are separated by a third time period having a duration of hours or days.

20. The method of claim 1 wherein the CNS non-implant mode includes transcranial magnetic stimulation.
